(19) United States
(12) Patent Application Publication
(10) Pub. No.: US 2026/0275402 A1
(43) Pub. Date: Sep. 17, 2026

(54) METHODS FOR TAGGING MOLECULES

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Axel DELAMARRE, New York, NY (US); Iestyn WHITEHOUSE, New York, NY (US)

(21) Appl. No.: 19/167,728

(22) PCT Filed: Mar. 25, 2024
(86) PCT No.: PCT/US2024/021297

§ 371 Date: Sep. 22, 2025

Related U.S. Application Data
(60) Provisional application No. 63/454,271, filed on Mar. 23, 2023.
(60) Provisional application No. 63/457,355, filed on Apr. 5, 2023.

(51) Int. Cl. C12Q 1/6804; C12Q 1/6806

(52) U.S. Cl. CPC C12Q 1/6804; C12Q 1/6806

(57) ABSTRACT

The present invention is directed to a method for tagging molecules as described herein.

This application claims priority to U.S. Provisional Application No. 63/454,271, filed on Mar. 23, 2023, and U.S. Provisional Application No. 63/457,355, filed Apr. 5, 2023, the entire contents of each of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. R01 GM129058 awarded by the NIH. The government has certain rights in the invention.

### FIELD OF THE INVENTION

This invention is directed to methods for tagging proximal molecules and uses of the same.

### BACKGROUND OF THE INVENTION

Genomics assays that report protein occupancy or the three-dimensional arrangement of crosslinked and fragmented chromosomes can be used to understand how genomic information is accessed, copied, or repaired. While powerful, genomics methods only interrogate a small fraction of the available information in a sample. Important questions related to whether events are coincident or mutually exclusive; or the nature of cause-effect relationships are difficult or impossible to assay.

### SUMMARY OF THE INVENTION

Aspects of the invention are directed towards a method of tagging proximal molecules. In embodiments, the method comprises admixing a seed nucleic acid, a receptor nucleic acid, an RNA polymerase, a reverse transcriptase, and two or more molecules to be tagged. In embodiments, the seed nucleic acid conjugates to a first molecule and comprises a promoter, a tag (or unique molecular identifier), and an annealing sequence. In embodiments, the receptor nucleic acid conjugates to a second molecule, or the other extremity of the same molecule, and comprises a nucleic acid complementary to the annealing sequence. In embodiments, the method comprises incubating the admixture for a period of time sufficient to allow (a) transcription of the seed nucleic acid by the RNA polymerase, thereby producing an RNA fragment, (b) annealing of the RNA to the receptor nucleic acid, and (c) reverse transcription of the RNA fragment by the reverse transcriptase, thereby producing a cDNA, thereby tagging proximal molecules.

In embodiments, the promoter comprises a T7 promoter, T3 promoter, or an SP6 promoter.

In embodiments, the annealing sequences comprises between 1 and 30 nucleotides. For example, the annealing sequence comprises about 20 nucleotides.

In embodiments, the annealing sequence comprises a nucleic acid sequence selected from the group consisting of 5′AAAAACCACAAAA3′, 5′AAAAGGAGAAAAAGGGAAAGAA3′, 5′AAAAGGAGAAAAAAAAGA3′, 5′AAAAGGAGAAAAA3′, or 5′TTTTGGTGTTTTT3′.

In embodiments, the seed nucleic acid, receptor nucleic acid, or both, comprises one or more modified ribonucleotide.

In embodiments, the receptor nucleic acid can be conjugated to a nanobody.

In embodiments, the RNA polymerase comprises T7, T3, or SP6.

In embodiments, the reverse transcriptase is a recombinant M-MuLV reverse transcriptase, AMV, Protoscript I, Protoscript II, Protoscript III, Protoscript IV, Superscript, or Induro.

In embodiments, the annealing is dependent on the Tm of the complementary sequences.

In embodiments, the method is carried out at between 20-37° C. For example, the reaction is carried out at 30° C.

In embodiments, the method further comprises one or more molecular crowding agents. For example, the molecular crowding agent comprises poly(ethylene glycol), glycerol, Dextran, Ficoll, or BSA.

In embodiments, the molecule is a nucleic acid, protein, or protein complex. For example, the protein or protein complex comprises a histone or a nucleosome.

In embodiments, the method comprises one or more additional steps of: (a) protein and/or DNA crosslinking prior to admixing; and/or (b) protein and/or DNA fragmentation prior to admixing; and/or (c) protein and/or DNA repair and A-tailing; and/or (d) seed nucleic acid and/or receptor nucleic acid ligation. For example, the crosslinking agent comprises formaldehyde, disuccinimidyl glutarate, or both. For example, the fragmentation comprises enzymatic fragmentation or mechanical fragmentation. For example, enzymatic fragmentation comprises a nuclease. For example, the ligation agent comprises T4 DNA ligase.

In embodiments, the method comprises one or more additional steps of: (a) a protein digestion step, and/or (b) an amplification step, and/or (c) a library preparation step, and/or (d) a sequencing step.

Aspects of the invention are further drawn to a kit comprising the reagents for any one of the methods as described herein. In embodiments, the kit comprises a seed nucleic acid, a receptor nucleic acid, an RNA polymerase, and a reverse transcriptase.

Aspects of the invention are further drawn to a method of whole chromosome analysis, wherein the method comprises the molecular tagging method as described herein.

Aspects of the invention are further drawn to a method of mapping 3D genomic organization, wherein the method comprises the molecular tagging method as described herein.

Aspects of the invention are further drawn to a method of protein detection or quantitation, wherein the method comprises the molecular tagging method as described herein.

Aspects of the invention are directed towards a molecular tagging method. In embodiments, the method comprises providing a molecule comprising a nucleic acid sequence flanked by a receptor nucleic acid sequence and a seed nucleic acid sequence. In embodiments, the receptor nucleic acid sequence comprises a single stranded region that can anneal to an annealing sequence. In embodiments, the seed nucleic acid sequence comprises a promoter, a random tag, and the annealing sequence. In embodiments, the method comprises incubating the molecule with an RNA polymerase, thereby providing RNA fragments that anneal to the receptor nucleic acid sequence. In embodiments, the method comprises reverse transcribing the annealed RNA sequence, thereby copying the RNA into DNA.

In embodiments, the molecule comprises a nucleic acid or protein. For example, the nucleic acid is DNA or RNA. For example, the protein is a nanobody or an antibody.

In embodiments, the molecule comprises a nucleosome.

In embodiments, the method further comprises crosslinking the nucleosomes.

In embodiments, the method further comprises fragmenting the crosslinked nucleosomes.

In embodiments, fragmenting the crosslinked nucleosomes provides DNA ends that can ligate to the receptor nucleic acid sequence and the seed nucleic acid sequence.

In embodiments, fragmenting comprises enzymatic fragmentation or mechanical fragmentation.

In embodiments, enzymatic fragmentation comprises a nuclease. For example, the nuclease is MNase, Caspase Activated DNase (CAD), or DNase I.

In embodiments, the receptor nucleic acid sequence and the seed nucleic sequence each comprises a linker. For example, the linker comprises a thymine overhang. For example, the thymine overhang can ligate to an “A”-tailed nucleosome.

In embodiments, A-tailing comprises adding a non-templated nucleotide to the 3′ end of the DNA ends.

In embodiments, the method further comprises incubating the nucleic acid sequence with a receptor nucleic acid sequence, a seed nucleic acid sequence, optionally a linker nucleic acid sequence, or any combination thereof, thereby providing a nucleic acid sequence flanked by the receptor nucleic acid sequence, the seed nucleic acid, the linker nucleic acid sequence, or any combination thereof.

In embodiments, the annealing is dependent on the Tm of the complementary sequences.

In embodiments, the annealing sequences comprise between 1 and 30 nucleotides. For example, the annealing sequence is about 20 nucleotides.

In embodiments, the RNA polymerase comprises T7 RNA polymerase, SP6, or T3 RNA polymerase.

In embodiments, the reverse transcriptase comprises MMLV, AMV, Protoscript I, Protoscript II, Protoscript III, Protoscript IV, Superscript, or Induro.

In embodiments, the nucleic acid molecule can be sequenced.

In embodiments, the method further comprises an amplification step.

In embodiments, the method further comprises the preparation of an output library.

In embodiments, the method further comprises sequencing the nucleic acid molecule.

Aspects of the invention are further drawn to a therapeutic target identified by any one of the methods as described herein.

Still further, aspects of the invention are drawn to a kit comprising the reagents for any one of the methods as described herein.

Aspects of the invention are further drawn to a method of whole chromosome analysis, wherein the method comprises the molecular tagging method as described herein.

Still further, aspects of the invention are drawn to a method of mapping 3D genomic organization, wherein the method comprises the molecular tagging method as described herein.

Other objects and advantages of this invention will become readily apparent from the ensuing description.

### BRIEF DESCRIPTION OF THE FIGURES

FIG. 1 shows an embodiment of the Proximity Copy & Paste (PCP) reaction. The test molecule contains a Seed sequence (green) and Receptor sequence (orange). The test molecule is radiolabeled to allow detection of DNA molecules, and the reaction is analy zed by denaturing PAGE. The test molecule is resolved as two bands reflecting the two strands of DNA; the strand with ssDNA overhang for the Receptor is longer. The full PCP reaction results in the specific extension of the longer strand.

FIG. 2 shows a schematic depicting that molecules in proximity are preferentially tagged. Two Seeds are introduced into the same reaction. Each Seed produces a slightly different RNA that can tag the Receptor on either seed. Whether a Seed tags in cis or in trans can be determined by PCR. The results show that “blue” tags “blue” and “red” tags “red” showing that Seeds preferentially tag a molecule in cis.

FIG. 3 shows a schematic depicting how the PCP reaction can be used to tag interacting molecules in a cell.

FIG. 4 shows a schematic depicting detection and quantification of proteins of interest in crosslinked chromatin fragments (CCFs). Antibodies can be attached to Receptor DNA molecules. This can allow interacting proteins to be identified in a complex mixture.

FIG. 5 shows a schematic depicting the tagging of nucleosomes in a PCP reaction. A region of crosslinked chromatin is digested with nuclease; Receptor and Seed sequences are ligated to DNA extremities. Each Seed contains a different Unique Molecule Identifiers (UMI; shown as different colors). During the PCP reaction, RNA produced from each seed will tag Receptors in proximity to the seed (shaded ovals). Overlap between shaded areas can be identified and used to map relative proximity of individual Seed nucleic acids.

FIG. 6 shows data depicting details of oligonucleotide design for the PCP assay on chromatin. This figure shows how Receptors are tagged and amplified in the PCP reaction. PCR primers P5 and P7 are used to amplify library for Illumina sequencing.

FIG. 7 shows data depicting details of oligonucleotide design for the PCP assay on chromatin. This figure shows the Seed sequence on the right and Receptor sequence on left through the PCP reaction. PCR primers P5 and P7 are used to amplify library for Illumina sequencing.

FIG. 8 shows data depicting benchmarking PCP-C vs Micro C-XL. Pairwise interaction maps are shown for whole genome (left). The PCP-C method detects centromere interactions between chromosomes (red dots) with greater sensitivity than Micro CXL (blue arrow points to a single centromere interaction). At high resolution on right, PCP-C detects more intermediate range interactions (green arrows).

FIG. 9 shows a schematic depicting the PCP tagging reaction. Panel A provides a schematic showing the PCP reaction on a model substrate. In this example, the Acceptor is in cis. The reaction requires both T7 RNA polymerase and MMLV reverse transcriptase for completion. Panel B shows a test of the PCP reaction using a radiolabeled seed/acceptor pair as shown in Panel A. The reaction contains dNTP and rNTP along with MMLV and/or T7 RNA polymerase, as shown. The reaction products are resolved on a denaturing PAGE (7M urea) gel. Strand-2 contains the annealing sequence and therefore is longer than strand-1. A specific extension product of strand-2 is formed with inclusion of both reverse transcriptase and T7 RNA polymerase. M is size marker.

FIG. 10 shows a schematic depicting the PCP reaction on chromatin. Panel A shows chromatin is crosslinked and digested with CAD nuclease, end-repaired and A-tailed; nucleosomes are white circles. Panel B shows linkers and Seeds are added and ligated to the ends of nucleosomes; individual Seeds with different UMIs are shown as colored nucleosomes. Panel C shows PCP reaction occurs on the chromatin, tagging RNAs diffusing from seeds are shown as large colored ovals. Panel D shows acceptor-linkers on specific nucleosomes near Seeds are tagged and shown as colored nucleosomes. Nucleosomes tagged by two adjacent Seeds (hetero-tagged) have two colors.

FIG. 11 shows PCP cis vs trans. Top panel provides a schematic depicting the PCP reaction containing two PCP substrates in competition: each seed is different (green and blue color), but the annealing sequence is the same (orange) allowing tagging in cis or trans. Middle panel shows that, using the two substrates, there four possible outcomes (illustrated), the presence of each product can be detected by PCR using specific primers (shown as blue or green arrows). Bottom panel shows products of the PCR reaction are resolved on agarose gel, primer pairs are shown above each lane. Specific product is seen with blue+blue or green+green primer pairs indicating cis tagging.

FIG. 12 shows data depicting an example of chromatin digestion by CAD or MNase at different concentrations (as indicated) and linker ligation. After end repair and A-tailing, linkers were ligated to chromatin. Following ligation, chromatin was digested with protease, the DNA was purified and analyzed on a native agarose gel. Linkers are efficiently ligated after CAD nuclease digestion; ovals indicate if 2 (fully shaded oval), 1 (half shaded oval) or 0 (not shaded oval) linkers are ligated to each nucleosome.

FIG. 13 shows a schematic depicting the overview of the PCP reaction on crosslinked nucleosomes. Top to Bottom: Chromatin is crosslinked and digested with CAD. DNA ends are repaired and A-tailed. A specific ratio of linkers is added and ligated to ends of DNA. Unligated linkers are removed and Seeds are ligated. Unligated Seeds are removed. PCP reaction takes place with limited amount of chromatin input. Chromatin is de-crosslinked and proteins are removed. PCR reaction is performed on de-proteinized DNA using primers compatible with Illumina machines. After limited cycles, the library (L) can be visualized by agarose gel electrophoresis.

FIG. 14 shows data depicting a comparison between PCP and Micro-C. Panel A shows a pairwise interaction map showing the 16 chromosomes from budding yeast. Top right is PCP data, bottom left is Micro-C. Interacting regions are shown as red pixels, the intensity scales with increasing interaction frequency. Note the centromeres on each chromosome cluster and give rise to distinct dots in the matrix. Panel B shows the interaction map of Panel A except a smaller region is shown. Note that PCP detects long-range interactions missed by Micro-C. Blue oval highlights interactions between HMR and MAT loci as part of mating type switching. Panel C shows the interaction map of Panel B, but showing chromatin folding in mitosis. ChIP of cohesin and condensin is shown for reference. PCP detects interactions not seen by Micro-C, as highlighted by yellow arrows. Panel D shows the interaction map as Panel B but at high resolution, showing interaction of individual genes. Individual nucleosomes are evident in the PCP data. Panel E shows cumulative interaction frequencies for PCP and Micro-C. PCP detects longer-range interactions than Micro-C. Panel F shows the number of nucleosomes tagged by each seed for a typical PCP reaction.

FIG. 15 shows a schematic depicting a dual-tagged nucleosome. Chromatin is ligated with a linkers and seeds for a PCP reaction. In this case several different linkers are used that contain short sequence variations (shown as yellow and green). After the PCP reaction, a nucleosome is tagged with RNA from two different seeds—Tag1 & Tag2 (shown as purple and brown, respectively). Pair-end sequencing will reveal the identity of the tag, the linkers and the genomic DNA. Dual tagged nucleosomes will have two different tags, but contain the same linker pairs and genomic DNA coordinates.

FIG. 16 shows a schematic depicting PCP-ID. Panel A shows chromatin is crosslinked and digested with CAD. Protein of interest is shown in green; nucleosomes are white ovals. Panel B shows linkers and seeds are ligated to nucleosomes; seeds ligated to nucleosomes are shown as blue and orange. Nanobody with linker is bound to protein of interest. Panel C shows the PCP reaction takes place on chromatin, linkers in proximity to seeds are tagged (shown as large colored ovals). The Nanobody is also tagged by the “blue” seed.

FIG. 17 shows data depicting purification and functionalization of a nanobody. Reaction components are resolved by SDS PAGE and silver-stained. Lane 1, Purified nanobody (N) is mixed with Sortase-A (S). Lane 2, oligonucleotide with 5 'azide (0). Lane 3, nanobody is mixed with Sortase, DBCO and oligo in ‘click’ reaction. The formation of the N—O product. Lane 4, MW marker. Lane 5-7, N—O product is bound to an affinity column and oligonucleotide is removed. Lane 8, N—O complex is eluted from the affinity matrix at high purity.

FIG. 18 shows a schematic depicting nanobody tagging in PCP. Panel A shows oligonucleotide containing specific sequences is bound to the nanobody using click reaction. Panel B shows that during PCP, the RNA tag anneals to the 3′ end of the oligo. Panel C shows that MMLV reverse transcriptase extends the free 3′ ends, copying the tag UMI into the oligo. Panel D shows that the tagged oligo can be amplified along with other tagged molecules in the PCP by PCR using primers compatible with Illumina.

FIG. 19 shows data depicting detection of transcription factor footprints with PCP. Top panel shows ChIP-seq data to map the position of the Rapl transcription factor (green signal, data depicted from Gutin et al). Middle panel shows pair-end PCP data was size selected to enrich sequenced molecules less than 70 nt. Plotting this data reveals specific footprints of transcription factors, such as Rapl (grey signal). Bottom panel shows PCP data for reference, the periodic pattern reflects positioned nucleosomes. Open reading frames are blue.

FIG. 20 shows data depicting the comparison of linker ligation efficiency after chromatin digestion by MNase vs CAD.

FIG. 21 shows data depicting the optimization of the annealing motif. Data showing the efficiency of the PCP reaction on a different model template with increasing T-tail on the receptor is provided.

FIG. 22 shows data depicting the design for new seeds. Efficiency of the PCP reaction on different annealing motifs is provided.

FIG. 23 shows data depicting the efficiency of the PCP reaction over time and with different Reverse-Transcriptases.

FIG. 24 shows data depicting the PCP competition reaction to test cis vs trans tagging.

FIG. 25 shows data depicting genomic tagging distance for PCP in relation to Micro-C and at different Seed: Receptor ratios.

FIG. 26 shows data depicting the comparison between PCP and Micro-C in mouse Embryonic Stem Cells (mESCs). PCP recapitulates the 3D features detected by Micro-C at several scales as viewed with Juicebox.

FIG. 27 shows a schematic depicting an overview of an experiment to model the detection and quantitation of protein binding to substrate using PCP. Panel A shows that the DNA substrate (Seed DNA) contains a single binding site (Rap1BS) for the yeast Rap1 transcription factor, a ‘Seed’ and a receptor. Panel B shows three different Rap1 proteins are prepared with differing numbers of copies of the ALFA epitope tag: Rap1_notag; Rap1_1xALFA; Rap1_3xALFA. Panel C shows the ALFA tag is detected with a nanobody with a single-strand oligonucleotide receptor covalently attached. Panel D shows the three separate reactions that are prepared, each containing the same quantity of Seed DNA substrate, the same quantity of anti-ALFA Nanobody and the same quantity of one of the three Rap1 proteins. Competitor oligonucleotides are added into each reaction to prevent tagging in trans (FIG. 28). In each reaction the Rap1 protein will bind to the substrate DNA, and the nanobodies will bind to the ALFA epitope tags (Panel D). After PCP is conducted on each reaction, the amount of tagged nanobodies will reflect the number of Rap1 epitopes bound by the nanobody in vicinity of the seed, thus a greatest proportion of nanobodies will be tagged in the reaction containing the Rap1_3ALFA protein. The Seed DNA substrate will be tagged to the same extent in each of the reactions, serving as a control for PCP efficiency (Panel D).

FIG. 28 shows a schematic depicting a competitor oligonucleotide included in PCP reactions increases specificity of tagging. The competitor sequence is composed of the PCP receptor annealing sequence and so can anneal to the tagging RNA produced from the seed. The competitor will sequester tagging RNAs that diffuse from the seed, such that the molecules in proximity to the seed will be preferentially tagged in the PCP reaction. Nanobodies that are not bound to a Rap1 protein are not tagged by the PCP reaction due to the excess of competitor. The 3′ extremity of the competitor is blocked by a dideoxycytidine (ddC), preventing its elongation by reverse transcriptase in the PCP reaction.

FIG. 29 shows data depicting a PCP tagging reaction as outlined in FIG. 27 and FIG. 28. Both the seed DNA and the nanobody receptor can be tagged in the PCP reaction and amplified by PCR. The amount of PCR product (analyzed by a native agarose gel) is proportional to the amount of specific tagging in the PCP. Each reaction has the same quantity of proteins, but differing numbers of ALFA epitope tags on each Rap1 protein. The amount of PCR product shows that nanobodies are tagged in proportion to the amount of ALFA epitope tags on the Rap1 protein.

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

Aspects of the invention are drawn towards methods for tagging proximal molecules with specific DNA sequences. For example, the methods described herein can comprise admixing a seed nucleic acid, a receptor nucleic acid, a RNA polymerase, a reverse transcriptase, and one or more molecules to be tagged.

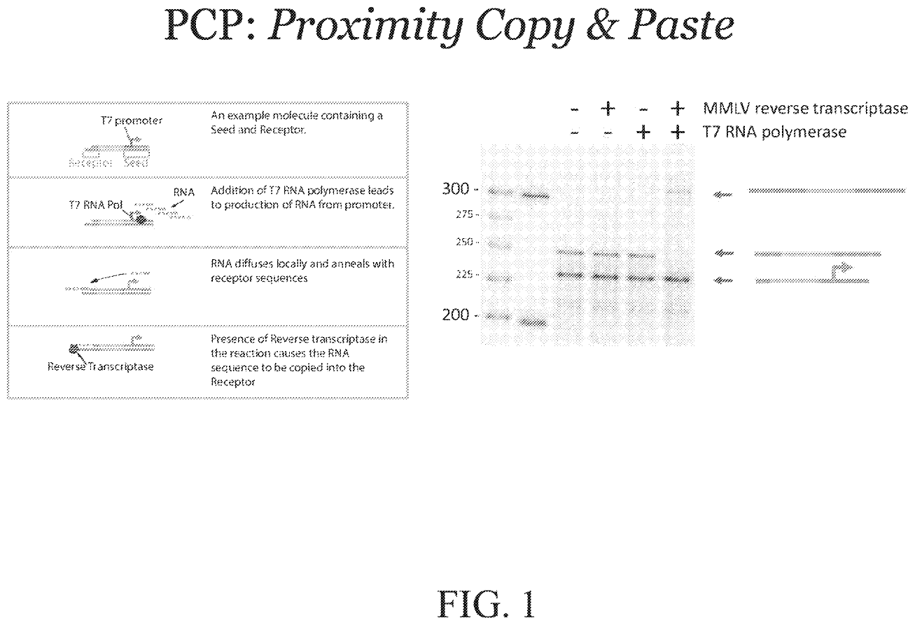

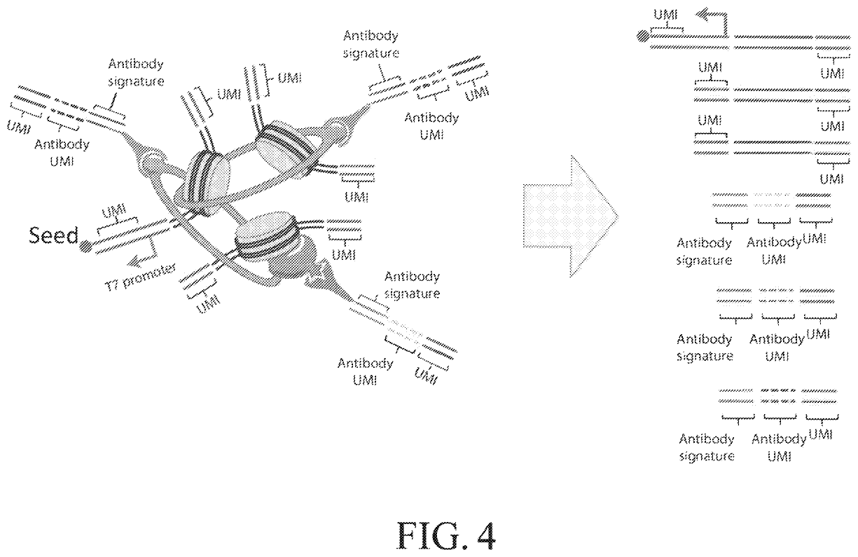

In embodiments, the seed nucleic acid conjugates to a first site and comprises a promoter, a tag (or unique molecular identifier (UMI)), and an annealing sequence. Further, the receptor nucleic acid conjugates to a second site, either on the same molecule as the first site or on a different molecule, and comprises a nucleic acid sequence complementary to the annealing sequence. The admixture can be incubated for a period of time sufficient to allow:
(a) transcription of the seed nucleic acid by the RNA polymerase, thereby producing an RNA fragment; (b) annealing of the RNA to the receptor nucleic acid, and (c) reverse transcription of the RNA fragment by the reverse transcriptase, thereby producing a cDNA. See, for example, FIG. 1 and FIG. 4.

Referring to FIG. 1, for example, an embodiment comprises providing a molecule, such as a nucleic acid molecule, comprising a nucleic acid sequence flanked by a receptor nucleic acid sequence and a seed nucleic acid sequence. The term “molecule” can refer to a substance composed of two or more atoms, or a group of like or different atoms held together by chemical forces (e.g., chemical bonds). For example, the molecule can comprise a nucleic acid, protein or protein complex. Referring to FIG. 4, for example, the seed nucleic acid sequence and the receptor nucleic action sequence comprise two or more different molecules. For example, the seed nucleic acid molecule comprises a promoter, a tag (or UMI), and an annealing sequence, and the receptor nucleic acid molecule comprises a nucleic acid sequence complementary to the annealing sequence.

In embodiments, the seed nucleic acid can be admixed with the receptor nucleic acid, either on the same or different nucleic acid molecules, and an RNA polymerase, a reverse transcriptase, and one or more molecules to be tagged.

The terms “Seed,” “seed nucleic acid sequence,” and “seed nucleic acid,” seed nucleic acid which can be used interchangeably, can refer to a nucleic acid that comprises a promoter that allows for the transcription by a single subunit RNA polymerase of a DNA sequence containing a region of variable DNA sequence (e.g., a Unique Molecule Identifier (UMI)) and an annealing sequence of known composition complementary to receptor DNA.

In embodiments, the Seed nucleic acid can attach to any protein, RNA, DNA or molecule of interest. For example, the Seed nucleic acid can attach to nucleosomal DNA by ligation, to RNA through ligation, to an antibody or nanobody through oligonucleotide-protein attachment chemistry, or can be integrated into DNA using a transposase, such as Tn5.

In embodiments, the Seed nucleic acid has a length of about 5 nucleotides, 10 nucleotides, 15 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, or 100 nucleotides. In some embodiments, the Seed nucleic acid can have a length of about 80 nucleotides.

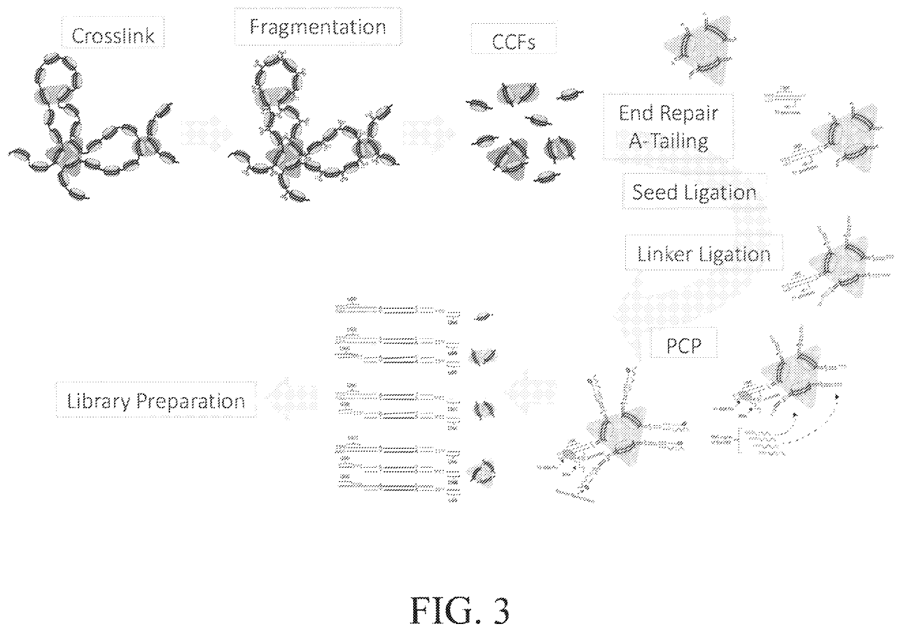

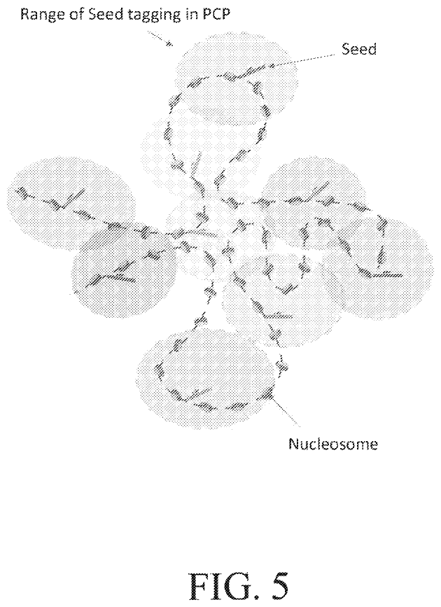

In embodiments, the Seeds nucleic acid contain a tag, otherwise referred to as a Unique Molecule Identifier (UMI). The term “Unique Molecule Identifier (UMI)” can refer to a type of molecular barcoding that can provide error correction and increased accuracy during sequencing. In embodiments, the UMI is random in order to give maximum diversity in the pool of seeds. In embodiments, the UMI can be designed to include a subset of sequences. In embodiments, UMIs can be engineered into the transcribed RNA. In embodiments, the UMI can provide that every Seed nucleic acid in the reaction produces multiple RNA of the same sequence. This method allows interacting molecules to be identified with single molecule precision. See, for example, FIG. 3 and FIG. 5.

The terms “Receptor,” “receptor nucleic acid sequence,” and “receptor nucleic acid”, which can be used interchangeably, can refer to a nucleic acid that comprises single strand DNA complementary to the annealing sequence on the RNA produced from the Seed. In embodiments, the Receptor can be a LNA. In embodiments, the Receptor nucleic acid can be RNA. In embodiments, the single strand DNA region of the Receptor nucleic acid has a length of about 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, or more than 30 nucleotides. For example, the single strand DNA region has a length of about 15 to 25 nucleotides.

In embodiments, the Seed nucleic acid transfers sequence information to the Receptor nucleic acid. In embodiments, the Receptor nucleic acid can be attached to any protein, RNA, DNA or molecule of interest in order to deduce its proximity to the Seed nucleic acid. In embodiments, the Receptor nucleic acid is on the same molecule as the seed nucleic acid. In embodiments, the Receptor nucleic acid attaches to a different molecule than the seed nucleic acid. In embodiments, the Seed nucleic acid and the Receptor nucleic acid are on the same nucleic acid. In embodiments, the Seed nucleic acid and the Receptor nucleic acid are on different nucleic acids. In embodiments, the receptor nucleic acid can comprise phosphorothioate bonds.

In embodiments, the seed nucleic acid and the receptor nucleic acid can be mixed at a seed to receptor ratio of about 1:5, 1:10, 1:25, 1:50, 1:100, 1:250, 1:500, 1:750, 1:1000, or more than 1:1000. For example, the seed nucleic acid and the receptor nucleic acid are mixed at a ratio of 1:20.

In embodiments, the method described herein further comprises one or more molecular crowding agents. The term “molecular crowding agent” or “crowding agent” can refer to an agent of a certain size that occupies space but does not interact with target proteins. Addition of a crowding agent can serve two purposes: 1) to stimulate annealing of the RNA to its target, such as to the Receptor nucleic acid, during the reaction; and 2) to regulate the diffusion of the RNA from the Seed nucleic acid to the Receptor nucleic acid during the reaction. Varying the amount of a crowding agent can influence the effective tagging distance between a Seed and Receptor in the molecular tagging method described herein. For example, the molecular crowding agent can comprise poly(ethylene glycol), glycerol, Dextran, Ficoll, or BSA.

A “nucleic acid” can refer to a polynucleotide sequence, or fragment thereof. The nucleic acid can comprise two or more nucleotides. The nucleic acid can be exogenous or endogenous to a cell. The nucleic acid can exist in a cell-free environment. The nucleic acid can be a gene or fragment thereof.

In embodiments, the nucleic acid can be DNA, which can refer to deoxyribonucleic acid. DNA can be double stranded including both complementary strands, unless the DNA is shown to be or indicated to be single stranded (ss) DNA.

In embodiments, the nucleic acid can be RNA, which can refer to a ribonucleic acid. RNA is a single stranded nucleic acid molecule, but can be a part of a double stranded molecule, for example, with complementary DNA (cDNA) by reverse transcription.

In embodiments, the nucleic acid can comprise one or more nucleic acid analogs (e.g., altered spine, sugar, or nucleobase). In some embodiments, the nucleic acid comprises a locked nucleic acid (LNA). A “locked nucleic acid” can refer to any bicyclic nucleic acid where a ribonucleoside is linked between the 2′-oxygen and the 4′-carbon atoms with a methylene unit.

In embodiments, the seed nucleic acid, the receptor nucleic acid, or both, can comprise one or more modified ribonucleotide. The term “ribonucleotide” and the phrase “ribonucleic acid” (RNA) can refer to a modified or unmodified nucleotide or polynucleotide comprising at least one ribonucleotide unit. For example, a modified ribonucleotide can comprise 2′-Fluoro RNA or 2′ methyl RNA.

The terms “polynucleotide” and “oligonucleotide” can be used interchangeably and can refer to a polymeric form of nucleotides of any length, and can include ribonucleotides, deoxyribonucleotides, analogs thereof, or mixtures thereof. The terms can be understood to include, as equivalents, analogs of DNA or RNA made from nucleotide analogs and to be applicable to single stranded (such as sense or antisense) and double stranded polynucleotides. The term as used herein also encompasses cDNA, that is complementary or copy DNA produced from an RNA template, for example by the action of reverse transcriptase. This term refers only to the primary structure of the molecule. Thus, the term includes triple-, double- and single-stranded deoxyribonucleic acid (“DNA”), as well as triple-, double- and single-stranded ribonucleic acid (“RNA”).

The terms “polypeptide,” “peptide” and “protein” can be used interchangeably herein to refer to a polymer of amino acid residues. In embodiments, the polymer can be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A polypeptide, or a cell, can be “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

In embodiments, the Seed nucleic acid and/or the Receptor nucleic acid can attach to one or more molecules. For example, the one or more molecules can comprise a protein or protein complex. For example, the protein or protein complex can comprise a histone or a nucleosome. The term “histone” can refer to a protein that provides structural support for a chromosome. Histones bind to DNA, help give chromosomes their shape, and help control the activity of genes. Eight histone proteins can come together to make up a nucleosome. The term “nucleosome” can refer to the basic repeating unit of chromatin. The human genome consists of several meters of DNA compacted within the nucleus of a cell having an average diameter of ~10 μm. In the eukaryote nucleus, DNA is packaged into a nucleoprotein complex known as chromatin. The nucleosome (the basic repeating unit of chromatin) can include ~146 base pairs of DNA wrapped approximately 1.7 times around a core histone octamer. The histone octamer consists of two copies of each of the histones H2A, H2B, H3 and H4. Nucleosomes are regularly spaced along the DNA in the manner of beads on a string.

In embodiments, the method described herein can be carried out at between 20-37° C. For example, the method is carried out at 30° C.

In embodiments, the Seed nucleic acid and/or the Receptor nucleic acid can be conjugated to an antibody. See, FIG. 4, for example. The term “antibody” can refer to an immunoglobulin, whether natural or partly or wholly synthetically produced. An antibody can include any immunoglobulin, including antibodies and fragments thereof, that binds a specific epitope. The term encompasses polyclonal, monoclonal, recombinant, humanized, and chimeric antibodies. The term also covers any polypeptide or protein having a binding domain which is, or is homologous to, an antibody binding domain. The term also encompasses CDR grafted antibodies. As antibodies can be modified in a number of ways, the term “antibody” can be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. An antibody fragment can bind to the same antigen that is recognized by the full-length antibody. An antibody fragment can include isolated fragments consisting of the variable regions of the antibodies, such as the “Fv” fragments consisting of the variable regions of the heavy and light chains and the recombinant single chain polypeptide molecules in which the light and heavy variable regions are connected by a peptide linker (“scFv proteins”). Exemplary antibodies can include, but are not limited to, antibodies to epitope tags (e.g., ALFA, FLAG, Myc, V5, SPOT, VHH, GFP, HA), antibodies to histone modifications, antibodies to nucleic acid binding proteins (e.g., transcription factors), cancer cell antibodies, virus antibodies, antibodies that bind to cell surface receptors (e.g., CDS, CD34, CD45), and therapeutic antibodies.

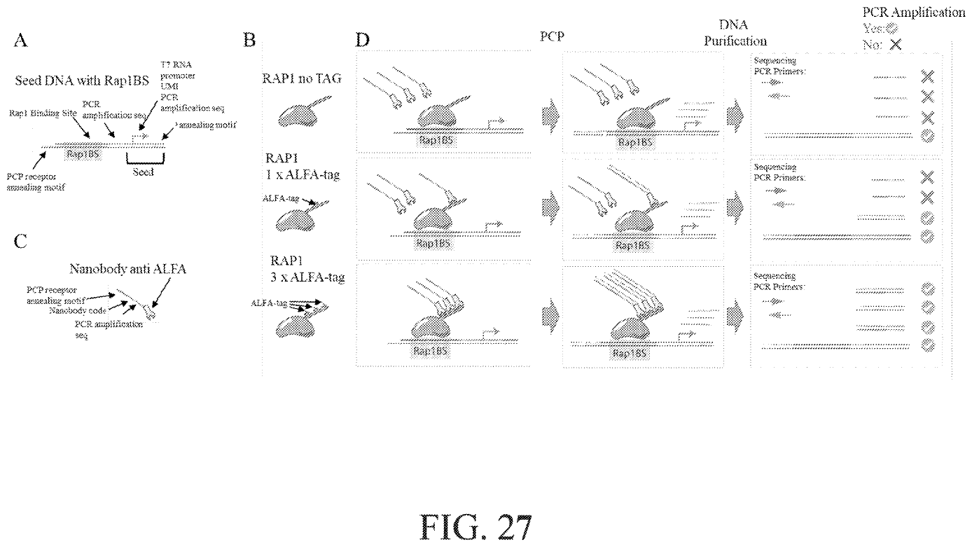

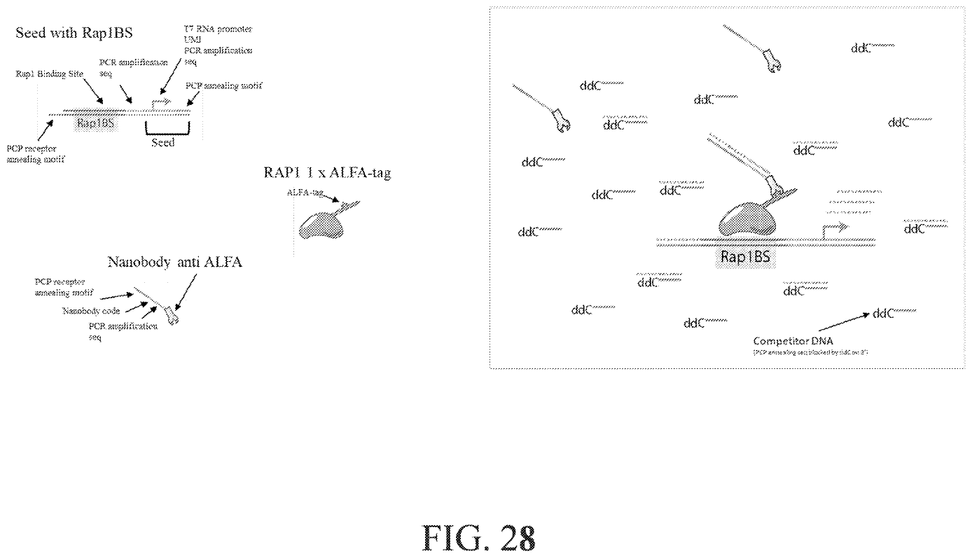

In embodiments, the Seed nucleic acid and/or the receptor nucleic acid can be conjugated to a nanobody. See FIG. 27, for example. A “nanobody” can refer to a single-domain antibody (sdAb), which is an antibody fragment consisting of a single monomeric variable antibody domain which is able to bind selectively to an antigen. A nanobody can comprise heavy chain variable domains or light chain variable domains. A nanobody can be derived from camelids (VHH fragments) or cartilaginous fishes (VNAR fragments). Alternatively, a nanobody can be derived from splitting the dimeric variable domains from IgG into monomers. Exemplary nanobodies can include, but are not limited to, nanobodies to epitope tags (e.g., ALFA, FLAG, Myc, V5, SPOT, VHH, GFP, HA) or post translational modification (e.g., phosphorylation, sumoylation, ubiquitination, methylation, acetylation). See, for example, FIG. 27 and FIG. 28.

In embodiments, the Seed nucleic acid and/or the receptor nucleic acid can be conjugated to a molecule that can be an engineered or natural protein with an affinity for specific targets, such as proteins that bind specific histone modifications (e.g., chromodomain binding to methylated histone tails), proteins that interact with specific nucleic acid sequences (e.g., CTCF), or subunits of larger protein complexes, such as the nuclear pore.

The term “primer” and its derivatives can refer to any nucleic acid that can hybridize to a target sequence of interest. In embodiments, the primer can function as a substrate onto which nucleotides can be polymerized by a polymerase; in some embodiments, however, the primer can become incorporated into the synthesized nucleic acid strand and provide a site to which another primer can hybridize to prime synthesis of a new strand that is complementary to the synthesized nucleic acid molecule. The primer can include any combination of nucleotides or analogs thereof. In some embodiments, the primer is a single-stranded oligonucleotide or polynucleotide.

In embodiments, the Seed nucleic acid can comprise a promoter. A “promoter” can refer to a control sequence that is a region of a nucleic acid sequence at which initiation and rate of transcription are controlled. It can contain genetic elements at which regulatory proteins and molecules can bind, such as RNA polymerase and other transcription factors, to initiate the specific transcription a nucleic acid sequence. The phrases “operatively positioned,” “operatively linked,” “under control,” and “under transcriptional control” can refer to a promoter that is in a correct functional location and/or orientation in relation to a nucleic acid sequence to control transcriptional initiation and/or expression of that sequence. For example, the promoter can comprise a T7 promoter, a T3 promoter, or an SP6 promoter.

In embodiments, the target molecule can be a nucleic acid or a protein.

In embodiments, the receptor nucleic acid sequence and/or the seed nucleic acid sequence can comprise a linker. The term “linker” can refer to an entity which links one or more molecules or molecule fragments to one or more tags. In embodiments, the linker has a length of about 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, or more than 30 nucleotides.

In embodiments, the linker has a length of about 1 base pair, 2 base pairs, 3 base pairs, 4 base pairs, 5 base pairs, 6 base pairs, 7 base pairs, 8 base pairs, 9 base pairs, 10 base pairs, 11 base pairs, 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs, 24 base pairs, 25 base pairs, 26 base pairs, 27 base pairs, 28 base pairs, 29 base pairs, 30 base pairs, or more than 30 base pairs.

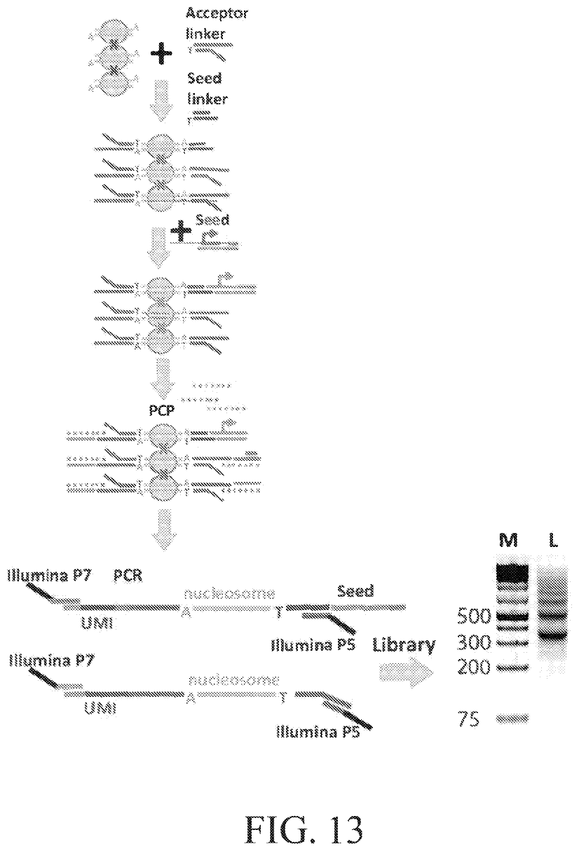

In embodiments, a linker can be ligated to a seed (seed-linker). In embodiments, the linker can comprise a thymine overhang. In embodiments, the thymine overhang can allow the linkers to be ligated to an end-repaired and “A”-tailed nucleosome. See, for example, FIG. 13. For example, the thymine overhang can ligate to an “A”-tailed nucleosome. The term “A-tail” can refer to a chain of adenine nucleotides that is added to a messenger RNA (mRNA) molecule during RNA processing to increase the stability of the molecule.

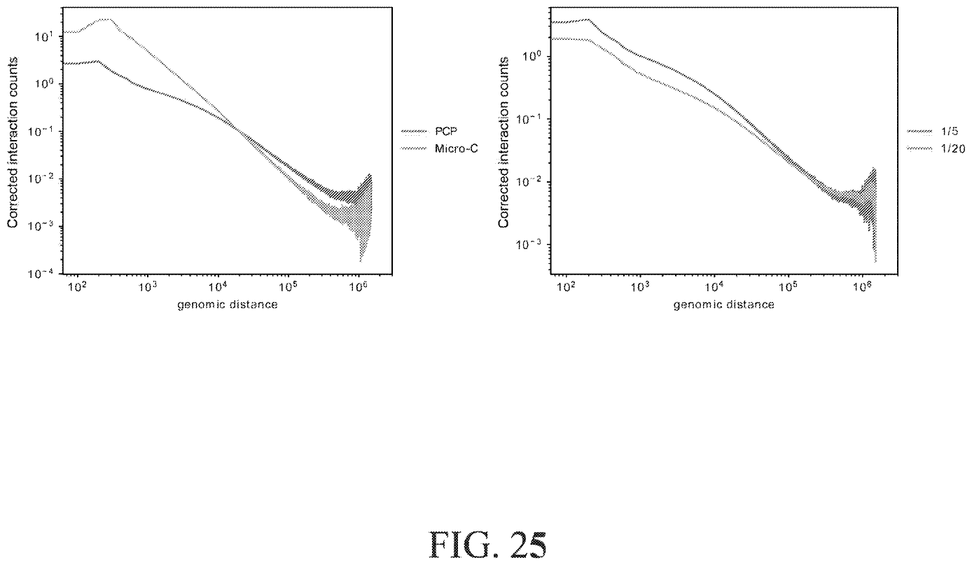

In embodiments, the methods described herein can tag molecules comprising a receptor nucleic acid in proximity or proximal to molecules comprising the Seed nucleic acid. The terms “proximity” or “proximal” can refer to nearness in distance, space, or relationship. See, for example, FIG. 25.

In embodiments, the receptor nucleic acid sequence comprises a single stranded region that anneals to an annealing sequence. The term “to anneal” or “annealing” can refer the formation of one or more complementary base pairs between two nucleic acids. In some embodiments, annealing involves two complementary or substantially complementary nucleic acids strands hybridizing together. In embodiments, in the context of an extension reaction, annealing involves the hybridization of primer to a template such that a primer extension substrate for a template-dependent polymerase enzyme is formed. In embodiments, conditions for annealing (e.g., between a primer and nucleic acid template) can vary based on the length and sequence of a primer. In embodiments, conditions for annealing are based upon a Tₘ (e.g., a calculated Tₘ) of a primer. In embodiments, the annealing sequence has a length of about 1 or more nucleotides, 5 or more nucleotides, 10 or more nucleotides, 15 or more nucleotides, 20 or more nucleotides, 25 or more nucleotides, 30 or more nucleotides, 35 or more nucleotides, 40 or more nucleotides, 45 or more nucleotides, 50 or more nucleotides, 100 or more nucleotides. In embodiments, the annealing sequence has a length of about 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, or more than 30 nucleotides. In embodiments, the annealing sequence has a length in range of about 1 to 5 nucleotides, 1 to 10 nucleotides, 1 to 15 nucleotides, 1 to 20 nucleotides, 1 to 25 nucleotides, 1 to 30 nucleotides, 1 to 35 nucleotides, 1 to 40 nucleotides, 1 to 45 nucleotides, or 1 to 50 nucleotides. In some embodiments, it can be useful to not utilize long poly-A motifs, as poly-A-tailed messenger RNA can compete with RNAs in the PCP reaction. For example, the annealing sequence can comprise the sequences 5′AAAAACCACAAAA 3′, 5′ AAAAGGAGAAAAAGGGAAAGAA 3′, 5′ AAAAGGAGAAAAAAAAGA 3′, 5′ AAAAGGAGAAAAA 3′, or 5′TTTTGGTGTTTTT 3′. For example, the annealing sequence can comprise about 20 nucleotides. In embodiments, conditions for annealing can be dependent on the Tm of the complementary sequences. In embodiments, the methods described herein can be carried out at between 20-37° C. For example, the reaction can be carried out at 30° C.

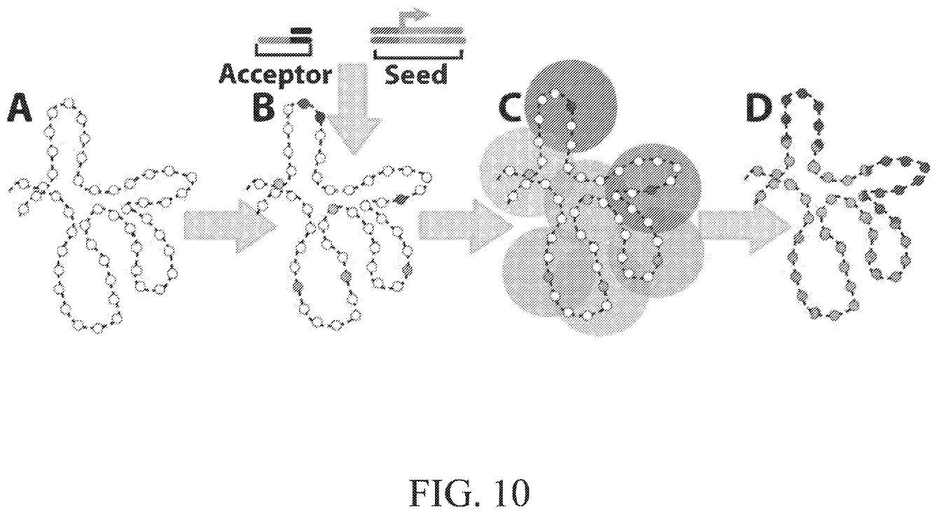

Embodiments can further comprise crosslinking the molecule, such that crosslinking agents can be used to preserve the 3D interactions of the molecule by creating covalent linkages between interacting proteins. For example, embodiments can further comprise crosslinking the nucleosomes and other cellular proteins, such that the 3D interactions of the nucleosomes and other proteins are maintained through nuclease digestion. See, for example, FIG. 10.

In embodiments, the cell or cell lysate can be treated with a crosslinker. The term “crosslinker” or “crosslinking agents” can refer to molecules that contain two or more reactive ends that chemically attach to specific functional groups on proteins or other molecules. The crosslinker can be added to the cell prior to cell lysis, or the crosslinker can be added to the cell lysate. Any suitable chemical crosslinker can be used. Non-limiting examples of chemical crosslinkers comprise formaldehyde, disuccinimidyl glutarate (DSG), ethylene glycol bis (succinimidyl succinate) (EGS), dimethyl adipimidate (DMA), dimethyl pimelimidate (DMP), dimethyl suberimidate (DMS), dimethyl dithiobispropionimidate (DTBP), and bis(sulfosuccinimidyl)suberate (BS3). In embodiments, disuccinimidyl glutarate (DSG) and/or formaldehyde crosslinkers can be used.

Embodiments can comprise fragmenting crosslinked genomes, nucleosomes, proteins (e.g., antibodies), and RNA. As used herein, “fragmenting” or “shearing,” and like terms, can refer to a process by which a larger molecule of DNA is converted into smaller pieces of DNA. For example, fragmenting can comprise enzymatic means, chemical means, or mechanical means of converting a large DNA molecule into a smaller one. For example, fragmenting of chromatin (e.g., chromosomal DNA) can be carried out using enzymatic means, mechanical means or chemical means. Non-limiting examples of mechanical shearing can include sonication or homogenization. Non-limiting examples of chemical shearing can include enzymatic fragmentation, using, for example a nuclease. A “nuclease” can refer to an enzyme that can degrade DNA or RNA by breaking phosphodiester bonds. Non-limiting examples of nucleases include Caspase Activated DNase (CAD), MNase, DNase I and Tn5.

Embodiments can comprise fragmenting the crosslinked nucleosomes, protein, or RNA to provide DNA ends that ligate to the receptor nucleic acid sequence and the seed nucleic acid sequence.

Embodiments can comprise fragmenting crosslinked genomes. As used herein, the term “genome” can refer to genomic information from a subject, which can be, for example, at least a portion or an entirety of a subject's hereditary information. A genome can be encoded in DNA or in RNA, or both. A genome can comprise coding regions (e.g., that code for proteins) as well as non-coding regions. A genome can include the sequence of the chromosomes together in an organism. For example, the human genome ordinarily has a total of 46 chromosomes. The sequence of these chromosomes together can constitute a human genome.

In embodiments, the admixture comprises a seed nucleic acid, a receptor nucleic acid, an RNA polymerase, a reverse transcriptase, and one or more molecules to be tagged. In embodiments, the admixture can be incubated for a period of time sufficient to allow: (a) transcription of the seed nucleic acid by the RNA polymerase, thereby producing an RNA fragment; (b) annealing of the RNA to the receptor nucleic acid; and (c) reverse transcription of the RNA fragment by the reverse transcriptase, thereby producing a cDNA. The terms “incubate,” “incubating,” or “incubation” can refer to the process of maintaining environmental conditions that are optimum for the reaction.

In embodiments, the reaction described herein can be incubated for about 1 minute, 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes 155 minutes, 160 minutes, 165 minutes, 170 minutes, 175 minutes, 180 minutes, about 3 hours, about 4 hours, about 5 hours, about 6 hours, about 7 hours, about 8 hours, about 9 hours, about 10 hours, about 11 hours, about 12 hours, or more. For example, the reaction can be incubated for about 60 minutes.

In embodiments, the reaction described herein can be incubated at about 1° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C. For example, the reaction can be incubated at about 20° C.

Embodiments further comprise admixing the molecule with an RNA polymerase, thereby providing RNA fragments that anneal to the receptor nucleic acid sequence. The term “RNA polymerases” can refer to an enzyme that catalyzes the polymerization of an RNA molecule. RNA polymerase can encompass DNA dependent as well as RNA dependent RNA polymerases. Non-limiting examples of RNA polymerases for use in the invention can include bacteriophage T7, T3 and SP6 RNA polymerases, E. coli RNA polymerase holoenzyme, E. coli RNA polymerase core enzyme, and human RNA polymerase I, II, III, human mitochondrial RNA polymerase and NS5B RNA polymerase from (HCV).

In cells, RNA polymerase is necessary for constructing RNA chains using DNA genes as templates, a process called transcription. RNA polymerase enzymes are essential to life and are found in all organisms and many viruses. RNA polymerase is a nucleotidyl transferase that polymerizes ribonucleotides at the 3′ end of an RNA transcript. Embodiments further comprise reverse transcribing the annealed RNA sequence, thereby providing a nucleic acid molecule that can be sequenced.

Embodiments further comprise admixing the molecule with a reverse transcriptase. The term “reverse transcriptases” can refer to a group of enzymes that have reverse transcriptase activity (i.e., they catalyze DNA synthesis from an RNA template). The term “reverse transcriptase activity” and “reverse transcription” can refer to the ability of an enzyme to synthesize a DNA strand (i.e., complementary DNA, cDNA) utilizing an RNA strand as a template. It is mainly associated with retroviruses. Non-limiting examples of a reverse transcriptase include M-MLV, M-MuLV, AMV, Protoscript I, Protoscript II, Protoscript III, Protoscript IV, Superscript, or Induro. One skilled in the art will be able to obtain commercially available reverse transcriptases that can be useful for the methods described herein.

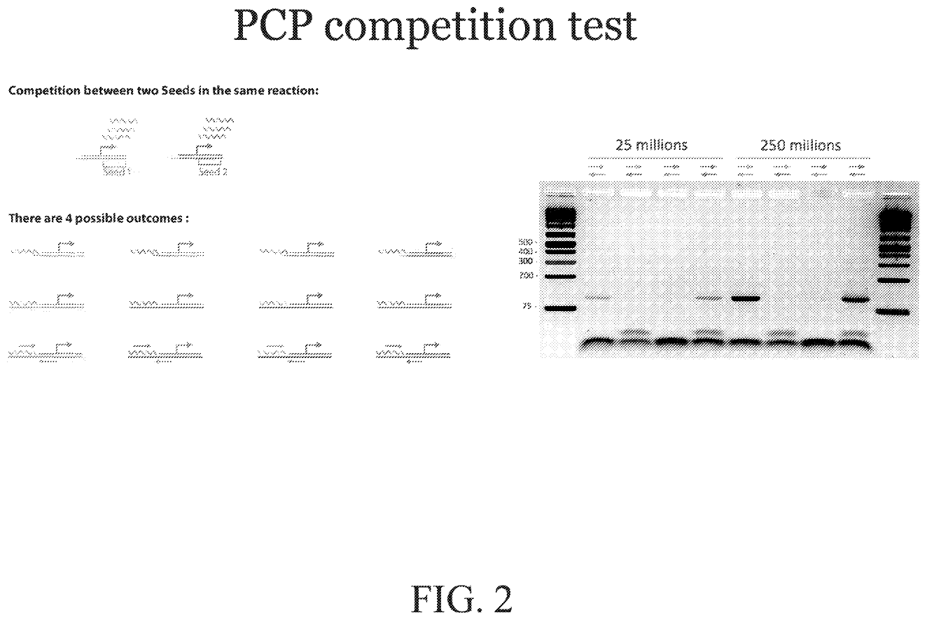

In embodiments, the method can be performed in a single step, for example mixing enzymes (e.g., T7 RNA polymerase and a reverse transcriptase in optimized buffer and conditions to attain maximal efficiency). See, for example, FIG. 2.

Embodiments can further comprise an amplification step. In embodiments, the term “amplification” can refer to a method that increases the number of copies of a molecule of DNA. Selective Amplification can refer to a method that increases the number of copies of a molecule of DNA, or molecules of DNA that correspond to a region of DNA. It can also refer to a method that increases the number of copies of a targeted molecule of DNA, or targeted region of DNA more than it increases non-targeted molecules or regions of DNA.

Components of an amplification reaction can include, but are not limited to, e.g., primers, a polynucleotide template, polymerase, nucleotides, dNTPs and the like. The term “amplifying” can refer to an “exponential” increase in target nucleic acid. However, “amplifying” as used herein can also refer to linear increases in the numbers of a select target sequence of nucleic acid, but is different than a one-time, single primer extension step.

In embodiments, amplification can be performed using a polymerase chain reaction (PCR). “Polymerase chain reaction” or “PCR” can refer to a method whereby a specific segment or subsequence of a target double-stranded DNA, is amplified in a geometric progression. PCR is well known to those of skill in the art. PCR involves in vitro amplification of specific DNA sequences by simultaneous primer extension of complementary DNA strands. Non-limiting examples of PCR comprise RT-PCR, real-time PCR, nested PCR, quantitative PCR, multiplexed PCR, digital PCR and PCR of assembly.

Aspects of the invention can further comprise preparing an output library. The term “output library” or “library” can refer to a library of nucleic acid molecules generated by the amplified products of the reaction described herein and that can be sequenced. In embodiments, the output library can determine the seed density of the reaction as the seed-linkers are part of the sequencing reads.

The term “sequencing,” as used herein, can refer to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®), Pacific Biosciences (PacBio®), Oxford Nanopore®), or Life Technologies (Ion Torrent®). Alternatively or in addition, sequencing can be performed using nucleic acid amplification, polymerase chain reaction (PCR) (e.g., digital PCR, quantitative PCR, or real time PCR), or isothermal amplification. Such systems can provide a plurality of raw genetic data corresponding to the genetic information of a subject (e.g., human), as generated by the systems from a sample provided by the subject. In some examples, such systems provide sequencing reads (also “reads” herein). A read can include a string of nucleic acid bases corresponding to a sequence of a nucleic acid molecule that has been sequenced. In embodiments, systems and methods provided herein can be used with proteomic information. In embodiments, high-throughput sequencing is performed. The term “high-throughput sequencing” or “massively parallel sequencing” can refer to a collection of methods and technologies that can sequence DNA thousands/millions of fragments at a time. Aspects of the invention can further comprise sequencing the nucleic acid molecule.

Aspects of the invention can comprise methods for identifying interactions of DNA, RNA, and/or protein molecules in a cell.

In embodiments, a method for identifying interactions of DNA, RNA, and/or protein molecules in a cell can comprise lysing a cell to form a cell lysate.

In embodiments, DNA, RNA, and/or protein interactions can be identified using a whole cell lysate. The term “whole cell lysate” can refer to the preparation obtained after lysing a population of whole cells using certain chemical reagents and enzymes, or by osmotic or mechanical disruption.

In embodiments, DNA, RNA, and/or protein interactions can be identified using a fractionated cell lysate. The term “fractionated cell lysate” can refer to the preparation obtained after lysing cells in order to separate subcellular components, and isolate organelles and other subcellular components from one another. For example, molecular interactions can be analy zed using the cytosol and/or any of the organelles. In embodiments, the nucleus can be isolated from the cell lysate for analysis of molecular interactions.

In embodiments, a Receptor nucleic acid cannot be tagged again once the Receptor nucleic acid is tagged. Accordingly, the Seed nucleic acids in the reaction compete to tag nearby Receptor nucleic acids. Consequently, a given Receptor can be tagged by a transcript produced by the most proximal Seed. Embodiments as described herein can also be referred to as Proximity Copy & Paste (PCP).

The term “tag” can refer to a non-target nucleic acid component, such as DNA, which provides a means of addressing a nucleic acid fragment to which it is joined. For example, in embodiments, a tag comprises a nucleotide sequence that permits identification, recognition, and/or molecular or biochemical manipulation of the DNA to which the tag is attached (e.g., by providing a site for annealing an oligonucleotide, such as a primer for extension by a DNA polymerase, or an oligonucleotide for capture or for a ligation reaction or by attaching a UMI). The process of joining the tag to the DNA molecule is sometimes referred to herein as “tagging” and DNA that undergoes tagging or that contains a tag can be referred to as “tagged” (e.g., “tagged DNA”).” The tag can have one or more tag portions or tag domains. In embodiments, the tag can comprise an epitope tag. Non-limiting examples of epitope tags comprise ALFA, FLAG, Myc, V5, SPOT, VHH, GFP, and HA.

As used herein, the terms “tagging” and “nucleotide tagging” can refer to the coupling of oligonucleotides to DNA, RNA, and/or protein molecules in order to label molecules that are found to interact (directly or indirectly) in a complex. For the methods described herein, tagging can occur when the RNA produced from the seed anneals to a receptor nucleic acid. Tagging is completed when that RNA is copied into DNA by reverse transcriptase. Tagging can refer to the oligonucleotide label (tag) that identifies molecules that sort together thereby receiving the same tag. Additionally, coupling of oligonucleotides, according to embodiments of the invention, can also be used to allow molecules to be tagged. For example, a protein or antibody can be coupled with an oligonucleotide in order for the protein or antibody molecule to subsequently receive (e.g., ligate) a nucleotide tag or receive a protein phosphate modified (PPM) adaptor that ligates a nucleotide tag. The coupling of oligonucleotides to proteins or antibodies is shown herein, but is also described in Los et al., “HaloTag: a novel protein-labeling technology for cell imaging and protein analysis, ACS Chem Biol., 2008, 3:373-382; Singh et al., “Genetically Encoded Multispectral Labeling of Proteins with Polyfluorophores on a DNA Backbone,” J. Am. Chem. Soc., 2013, 16:6184-6191; Blackstock et al., “Halo-Tag Mediated Self-Labeling of Fluorescent Proteins to Molecular Beacons for Nucleic Acid Detection,” Chem. Commun., 2014, 50:1375-13738; Kozlov et al., “Efficient Strategies for the Conjugation of Oligonucleotides to Antibodies Enabling Highly Sensitive Protein Detection,” Biopolymers, 2004, 73:621; and Solulink, “Antibody-Oligonucleotide Conjugate Preparation,” Solulink.com, 4 pages, the entire contents of all of which are incorporated herein by reference.

The term “molecular tag” can refer to a molecule that can bind to a macromolecular constituent. The molecular tag can bind to the macromolecular constituent with high affinity. The molecular tag can bind to the macromolecular constituent with high specificity. The molecular tag can comprise a nucleotide sequence. The molecular tag can comprise a nucleic acid sequence. The nucleic acid sequence can be at least a portion or an entirety of the molecular tag. The molecular tag can be a nucleic acid molecule or can be part of a nucleic acid molecule. The molecular tag can be an oligonucleotide or a polypeptide. The molecular tag can comprise a DNA aptamer. The molecular tag can be or comprise a primer. The molecular tag can be, or comprise, a protein. The molecular tag can comprise a polypeptide. The molecular tag can comprise a barcode. As used herein, “adding,” and like terms, can refer to the combination of two or more components together, no matter the order of the addition. For example, “adding” a nucleotide tag to a molecule is the same as “adding” a molecule to a nucleotide tag so long as the nucleotide tag and the molecule are combined.

Aspects of the invention are further drawn to methods of identifying a therapeutic target using methods described herein. The term “therapeutic target” can refer to a native protein, molecule, compound, nucleic acid, organ, gland, ligand, receptor, organelle, or cell whose activity is modified by a drug resulting in a desirable therapeutic effect.

Aspects of the invention are further drawn to methods of whole chromosome analysis using methods described herein. The term “whole chromosome analysis” can refer to a test that evaluates the number and structure of a chromosomes in order to detect abnormalities. For example, the method can comprise providing a molecule comprising a nucleic acid sequence flanked by a receptor nucleic acid sequence and a seed nucleic acid sequence, wherein the receptor nucleic acid sequence comprises a single stranded region that can anneal to an annealing sequence; and wherein the seed nucleic acid sequence comprises a promoter, a random tag, and the annealing sequence; incubating the molecule with an RNA polymerase, thereby providing RNA fragments that anneal to the receptor nucleic acid sequence; and reverse transcribing the annealed RNA sequence, thereby providing a nucleic acid molecule.

Aspects of the invention are further drawn to methods of mapping chromatin DNA complexes using methods described herein. The term “mapping” can refer to the process of determining the relative locations of landmarks or markers (e.g., proteins binding sites, variants and other DNA sequences of interest) within a chromosome or genome. The term “chromatin DNA complexes” can refer to the complex of DNA and protein found in eukaryotic cells. For example, the method can comprise providing a molecule comprising a nucleic acid sequence flanked by a receptor nucleic acid sequence and a seed nucleic acid sequence, wherein the receptor nucleic acid sequence comprises a single stranded region that can anneal to an annealing sequence; and wherein the seed nucleic acid sequence comprises a promoter, a random tag, and the annealing sequence; incubating the molecule with an RNA polymerase, thereby providing RNA fragments that anneal to the receptor nucleic acid sequence; and reverse transcribing the annealed RNA sequence, thereby providing a nucleic acid molecule.

Aspects of the invention are further drawn to methods of mapping three-dimensional nucleic acid organization (e.g., genomic organization) using methods described herein. For example, the method can comprise providing a molecule comprising a nucleic acid sequence flanked by a receptor nucleic acid sequence and a seed nucleic acid sequence, wherein the receptor nucleic acid sequence comprises a single stranded region that can anneal to an annealing sequence; and wherein the seed nucleic acid sequence comprises a promoter, a random tag, and the annealing sequence; incubating the molecule with an RNA polymerase, thereby providing RNA fragments that anneal to the receptor nucleic acid sequence; and reverse transcribing the annealed RNA sequence, thereby providing a nucleic acid molecule.

In embodiments, a method for identifying interactions of DNA, RNA, and/or protein molecules in a cell can comprise lysing a cell to form a cell lysate.

In embodiments, DNA, RNA, and/or protein interactions can be identified using a whole cell lysate. The term “whole cell lysate” can refer to the preparation obtained after lysing a population of whole cells using certain chemical reagents and enzymes, or by osmotic or mechanical disruption.

The term “sample” can refer to a quantity of biological molecules that are to be tested for the presence or absence of one or more molecules. The sample can comprise any number of macromolecules, for example, cellular macromolecules. The sample can be a nucleic acid sample or protein sample. The sample can also be a carbohydrate sample or a lipid sample. The sample can be derived from another sample. The sample can be a tissue sample, such as a biopsy, core biopsy, needle aspirate, or fine needle aspirate. The sample can be a fluid sample, such as a blood sample, urine sample, or saliva sample. The sample can be a skin sample. The sample can be a cheek swab. The sample can be a plasma or serum sample. The sample can be a cell-free or cell free sample. A cell-free sample can include extracellular polynucleotides. Extracellular polynucleotides can be isolated from a bodily sample that can be selected from the group consisting of blood, plasma, serum, urine, saliva, mucosal excretions, sputum, stool and tears.

The term “test sample” can refer to a sample in which the presence or amount of one or more analytes of interest are unknown and to be determined, such as using methods as described herein. In embodiments, a test sample can be a bodily fluid obtained for the purpose of diagnosis, prognosis, or evaluation of a subject, such as a patient. In embodiments, such a sample can be obtained for the purpose of determining the outcome of an ongoing condition or the effect of a treatment regimen on a condition. Non-limiting examples of such test samples can include blood, serum, plasma, cerebrospinal fluid, urine and saliva. Some test samples are more readily analyzed following a fractionation or purification procedure, for example, separation of whole blood into serum or plasma components. In embodiments, samples can be obtained from bacteria, viruses and animals, such as dogs and cats. In embodiments, samples can be obtained from humans. By way of contrast, a “standard sample” can refer to a sample in which the presence or amount of one or more analytes of interest are known prior to assay for the one or more analytes. Some test samples obtained from patients can be referred to as “test samples.”

The term “disease sample” can refer to a tissue sample obtained from a subject that has been determined to suffer from a given disease. Methods for clinical diagnosis are well known to those of skill in the art. See, e.g., Kelley's Textbook of Internal Medicine, 4th Ed., Lippincott Williams & Wilkins, Philadelphia, Pa., 2000; The Merck Manual of Diagnosis and Therapy, 17th Ed., Merck Research Laboratories, Whitehouse Station, N.J., 1999. “Disease” includes events accepted in the medical field as adverse outcomes related to a disease, such as stroke, myocardial infarction, and other adverse health events.

The term “biological particle” can refer to a discrete biological component derived from a biological sample. The biological particle can be a virus. The biological particle can be a cell or derivative of a cell. The biological particle can be an organelle. The biological particle can be a rare cell from a population of cells. The biological particle can be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle can be or can include a matrix (e.g., a gel or polymer matrix) comprising a cell or one or more constituents from a cell (e.g., cell bead), such as DNA, RNA, organelles, proteins, or any combination thereof, from the cell. The biological particle can be obtained from a tissue of a subject. The biological particle can be a hardened cell. Such hardened cell can or cannot include a cell wall or cell membrane. The biological particle can include one or more constituents of a cell, but cannot include other constituents of the cell. An example of such constituents is a nucleus or an organelle. A cell can be a live cell. The live cell can be cultured, for example, being cultured when enclosed in a gel or polymer matrix, or cultured when comprising a gel or polymer matrix.

As used herein, the term “cell” can refer to one or more cells. In embodiments, the cells can be normal cells (e.g., human cells at different stages of development), or human cells from different organs or tissue types (e.g., white blood cells, red blood cells, platelets, epithelial cells, endothelial cells, neurons, glial cells, fibroblasts, skeletal muscle cells, smooth muscle cells, gametes or cells of the heart, lungs, brain, liver, kidney, spleen, pancreas, thymus, bladder, stomach, colon, small intestine). In embodiments, the cells can be undifferentiated human stem cells, or human stem cells that have been induced to differentiate. In embodiments, the cells can be human fetal cells. Human fetus cells can be obtained from a pregnant mother of the fetus. In embodiments, the cells are rare cells. A rare cell can be, for example, a circulating tumor cell (CTC), a circulating epithelial cell, a circulating endothelial cell, a circulating endometrial cell, a circulating stem cell, a stem cell, an undifferentiated stem cell, a cancer stem cell, a bone marrow cell, an immune cell, a progenitor cell, foam cell, mesenchymal cell, trophoblast, immune system cell (host or graft), cell fragment, cell organelle (e.g., mitochondria or nuclei), pathogen-infected cell, and the like.

In embodiments, the cells can be non-human cells, e.g., other types of mammalian cells (e.g., mouse, rat, pig, dog, cow, or horse). In embodiments, the cells are other types of animal or plant cells. In embodiments, the cells can be any prokaryotic or eukaryotic cell. For example, the cells can be yeast cells.

In embodiments, a first sample of cells is obtained from a person who does not have a disease or condition, and a second sample of cells is obtained from a person who has the disease or condition. In some embodiments, people are different. In some embodiments, the people are the same but the cell samples are taken at different time points. In some embodiments, people are patients, and the cell samples are patient samples. The disease or condition can be a cancer, a bacterial infection, a viral infection, an inflammatory disease, a neurodegenerative disease, a fungal disease, a parasitic disease, a genetic disorder, or any combination of these.

The terms “individual”, “patient” and “subject” can be used interchangeably. They can refer to a mammal (e.g., a human) which is the object of treatment, or observation. Typical subjects to which compositions and methods described herein can be administered will be mammals, such as primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals, such as dogs and cats. For diagnostic or research applications, a wide variety of mammals will be suitable subjects, including rodents (e.g., mice, rats, hamsters), rabbits, primates, and swine such as inbred pigs and the like.

Aspects of the invention are also directed towards kits, such as kits comprising the reagents as described herein for molecular tagging.

In one embodiment, the kit includes (a) a container that contains the reagents(s), such as that described herein, and optionally (b) informational material. The informational material can be descriptive, instructional, marketing or other material that relates to the methods described herein and/or the use of the agents for research and/or therapeutic benefit.

The informational material of the kits is not limited in its form. In one embodiment, the informational material can include information about production of the reagents, molecular weight of the reagents, concentration, date of expiration, batch or production site information, and so forth. The information can be provided in a variety of formats, include printed text, computer readable material, video recording, or audio recording, or information that provides a link or address to substantive material.

The kit can include other ingredients, such as a solvent or buffer, a stabilizer, or a preservative. When the reagents are provided in a liquid solution, the liquid solution can be an aqueous solution. When the reagents are provided as a dried form, reconstitution is by the addition of a suitable solvent. The solvent, e.g., sterile water or buffer, can optionally be provided in the kit.

The kit can include one or more containers for the reagents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a target ratio. For example, the kit includes a plurality of syringes, ampules, foil packets, blister packs, or medical devices, e.g., each containing a single combination unit dose. The containers of the kits can be air-tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Proximity Copy & Paste (PCP)

### Overview of Embodiments of the Invention

The Proximity Copy & Paste (PCP) reaction is a new method for tagging molecules with specific DNA sequences.

In the PCP reaction, a sequence of DNA is copied from a ‘Seed’ molecule to a ‘Receptor’ molecule. The Seed comprises a promoter that will allow for the transcription of a DNA sequence by a single subunit RNA polymerase e.g., T7 RNA polymerase. At the end of the transcribed DNA is a specific sequence that will allow the RNA to anneal to Receptor nucleic acids. Upon annealing, this transcript can be used as a template by a Reverse Transcriptase, resulting in the elongation (tagging) of the 3′ of the receptor with the transcript sequence from the Seed (FIG. 1).

The PCP reaction results in the transfer of sequence information from the Seed to Receptor(s). RNA polymerases can transcribe any DNA sequence, so a large variety of DNA sequences can be engineered into the Seed. Furthermore, the transcription process makes many RNA copies of the same sequence, which allows multiple receptor nucleic acids to be tagged with a single Seed.

The receptor nucleic acid can be located on the same molecule as the Seed (in cis) or on other molecules in the reaction (in trans). Importantly, the PCP reaction can tag Receptor nucleic acids in proximity to the Seed. In addition, the reaction can be performed in a single step mixing both enzymes in optimized buffer and conditions to attain maximal efficiency (FIG. 2).

Once a Receptor is tagged, it cannot be tagged again. The Seeds in the reaction therefore compete to tag nearby Receptors. Consequently, a given Receptor can be tagged by a transcript produced by the most proximal Seed.

The PCP reaction is highly amenable to high throughput sequencing. Seeds can contain any sequence of interest; Unique Molecule Identifiers (UMI) are engineered into the transcribed RNA. The UMI ensures that every Seed in the reaction produces a unique RNA so every Receptor nucleic acid tagged by an individual Seed can be identified by reading the UMI attached to it. This method therefore allows interacting molecules to be identified with single molecule precision (FIG. 3).

A variety of molecules can be engineered to participate in the PCP reaction. For example, if an antibody against a protein of interest is conjugated with a Receptor DNA molecule, then the presence of the antibody in proximity to a seed can be deduced using the PCP method (FIG. 4). In embodiments, a Receptor can be attached to any protein, RNA, DNA or molecule of interest and deduce its proximity to the Seed. Similarly, the Seed can be attached to any protein, RNA, DNA or molecule of interest.

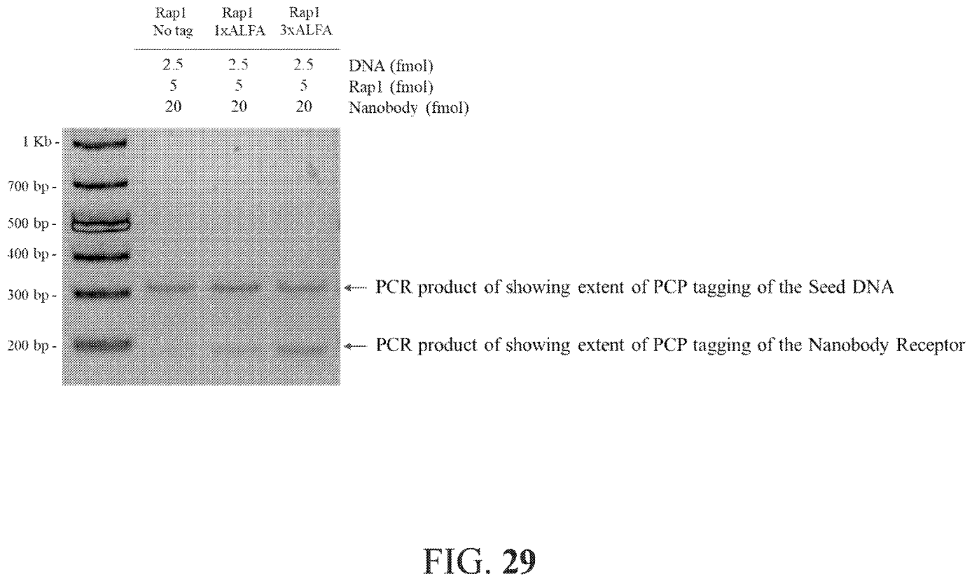

Quantification of the amount of interacting molecules can be accomplished with use of specific DNA sequences, barcodes or UMI's engineered into the Seed and Receptor nucleic acids. For example, if an antibody is conjugated with a Receptor nucleic acid, and each Receptor contains a UMI, the number of unique Receptor nucleic acids tagged by each unique Seed in the PCP reaction will indicate the number of antibodies in proximity to the seed. See, for example, FIG. 27 and FIG. 29.

Individual molecules can be engineered to contain two or more Receptors. For example, a mono-nucleosome has two DNA ends. If both ends contain a Receptor, the PCP reaction can result in each Receptor being tagged with RNA from the same Seed. It is also possible that the two Receptors on a single mono-nucleosome can be tagged with RNA from different seeds, this will indicate that two different seeds are near the mono-nucleosome. Using this logic, the relative spatial relationship between two (or more) Seeds and Receptors can be deduced (FIG. 5).

### Specifics of the PCP Reaction.

The PCP reaction currently utilizes the single subunit T7 RNA polymerase with an efficient promoter encoded into the Seed. Other RNA polymerases, such as SP6 can produce similar results.

The 3′ end of the RNA molecules produced can contain a sequence that can anneal to the single-strand part of the Receptor nucleic acid. The RNA can be any sequence of interest; we have found the following sequence to be optimal for the 3′ end of the RNA to anneal to a complementary Receptor: 5′AAAAACCACAAAA 3′. Other examples include: 5′ AAAAGGAGAAAAAGGGAAAGAA 3′, 5′ AAAAGGAGAAAAAAAAGA 3′, 5′ AAAAGGAGAAAAA 3′, 5′ TTTTGGTGTTTTT 3′.

Degradation of the RNA can inhibit the PCP reaction in cells, tissues and extracts, and, without wishing to be bound by theory, inclusion of modified ribonucleotides in the PCP reaction will stabilize the RNA and prevent degradation.

The PCP reaction currently utilizes MMLV reverse transcriptase. Any enzyme with Reverse Transcriptase activity will function in the PCP reaction. We have found that Protoscript II from NEB gives optimal results in the PCP reaction.

While T7 and MMLV have optimum temperature of 37° and above, we have found that the PCP reaction is currently most efficient at 30° C. The optimum temperature is strongly influenced by the efficiency with which the RNA from the Seed anneals with the Receptor. This annealing is dictated by the length of the homology between the RNA and the Receptor. Different reaction temperature can be used depending on the design of the RNA and buffer components.

The PCP reaction is stimulated by the addition of molecular crowding agents, such as poly(ethylene glycol) and glycerol. Addition of crowding agents serves two purposes: first, to stimulate annealing of the RNA to its target: second, to regulate the diffusion of the RNA from the Seed during the reaction. Decreased diffusion will increase local concentration of the RNA near the Seed and so limit tagging to receptors in proximity to the Seed. Varying the amount of crowding agent will influence the effective tagging distance between a Seed and Receptor in the PCP reaction.

RNA tags can diffuse from the Seed nucleic acid and can potentially tag any receptor nucleic acid in the reaction. This may result in random tagging where the Receptor nucleic has no spatial relationship with the Seed nucleic acid. Such random tagging is generally disfavored as Receptor nucleic acids close to the Seed nucleic acid are preferentially tagged (FIG. 2) but it can occur. Random tagging can be suppressed by inclusion of a competitor oligonucleotide that is able to anneal to the tagging RNA. When the competitor oligonucleotide anneals to the tagging RNA, it prevents the tagging RNA from annealing to a Receptor nucleic acid. Competitor oligonucleotides can be added to the PCP reaction at various concentrations and will further bias the tagging reaction towards Receptors in proximity to the Seed (FIG. 28).

### The PCP Reaction to Map Higher Order Chromatin Folding.

Chromatin packages the genome and plays critical roles in many DNA dependent transactions. The fundamental repeating unit of chromatin is the nucleosome. Each nucleosome is composed of a core of histones proteins around which approximately 150 bp of DNA is wrapped. Nucleosomes are found across eukaryotic genomes and their ability to interact with each other and with other proteins is critical in the regulation of the genome function. Genomes fold into complex structures in 3D space, these structures often involve DNA sequences that are separated by large genomic distances, coming into proximity in 3D space. Use of crosslinking agents such as formaldehyde can preserve these 3D interactions by creating covalent linkages between interacting proteins, such as histones.

Pioneering work has developed methods to map how chromatin folds in 3D space. These approaches—referred to as Chromatin Conformation Capture (3C)—rely on crosslinking chromatin to preserve 3D organization, then fragmenting the DNA with restriction enzymes or nucleases, and then ligating proximal DNA ends together. Sequencing across the ligation junctions allows the identity of two interacting DNA molecules to be deduced. 3C methods have limitations, primarily related to the inefficiency of the DNA ligation reaction.

More recent approaches rely on isolation and analysis of crosslinked chromatin fragments (CCFs). The SPRITE method utilizes crosslinking to preserve chromatin contacts and then immobilizes individual CCFs on magnetic beads. The DNA contained within each CCF is then uniquely indexed using a “split-pool” barcoding approach. The SPRITE method allows each CCF to be uniquely tagged and provides single molecule precision to interacting DNA (and RNA) molecules. A related method—ChIA-Drop—utilizes microfluidics provided in the 10X genomics system to encapsulate individual CCFs into lipid droplets, allowing the ends of DNA to be uniquely tagged. Both methods are potentially superior to 3C approaches as their output has single-molecule precision and is not limited to mapping pairwise interactions. However, both SPRITE and ChIA-Drop are difficult and require expensive reagents and instrumentation.

The PCP method offers significant advantages over 3C, SPRITE and ChIA-Drop methods for analysis of chromatin structure. PCP provides single molecule precision with a simple workflow that can be accomplished for a fraction of the time and cost of other approaches.

Overview of Method for Chromatin Structure Analysis with PCP-C.

### 1: Crosslinking.

Chromatin within cells is first crosslinked with a combination of formaldehyde and disuccinimidyl glutarate (a bifunctional crosslinking agent). Other crosslinking agents can be substituted depending on the extent of crosslinking required.

### 2: DNA Fragmentation.

Chromatin is digested with a nuclease to generate DNA ends for subsequent Receptor and Seed ligations. The choice of nuclease influences the efficiency and resolution of the assay. Micrococcal Nuclease (MNase) is used for chromatin digestion in high-resolution assays, such as Micro-C. We have found that MNase is not well suited for this purpose as the DNA ends generated by MNase digestion are often incapable of being ligated to other molecules. This stems from the fact that MNase leaves a terminal phosphate in a position incompatible with ligation by T4 DNA ligase; in addition, the exonucleolytic activity of MNase leaves DNA ends incompatible with enzymatic manipulation. The high-resolution PCP reaction utilizes a Caspase Activated DNase (CAD) that overcomes the above limitations with MNase. CAD digested chromatin is far more amenable to enzymatic DNA repair and subsequent ligation.

### 3: Ligation of Receptor and Seed-Linker Molecules.

In the current method, the DNA ends of CAD digested nucleosomes are repaired and A-tailed using commercial enzyme preparations (NEB ultra II end repair and A tail). The repaired chromatin is pelleted and washed to remove the enzymes of the repair kit. Next a mixture of “Receptor” and “Seed-Linker” oligonucleotides are added and ligated to the chromatin using T4 DNA ligase.

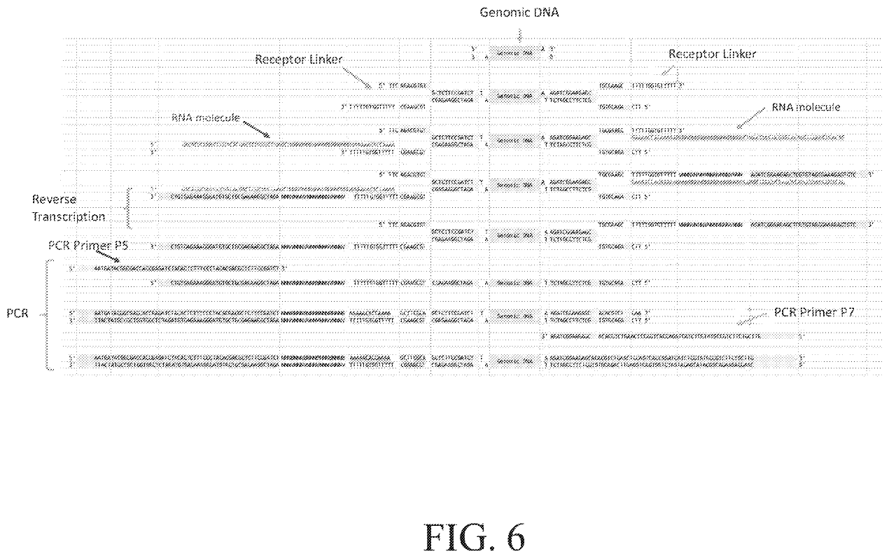

The Receptor nucleic acids contain a single 3′ T overhang at one end to ligate to the A-tailed chromatin, and the other end is a stretch of ssDNA complementary to the RNA produced from the seed. The Receptor nucleic acids also contain specific DNA sequences that allow amplification in PCR following the PCP reaction (FIG. 6).

Seed-Linker molecules contain a single 3′ T overhang at one end to ligate to the A-tailed chromatin, the opposite end of the molecule contains an overhang complementary to the Seed nucleic acid.

The ratio of Receptor to Seed-Linker molecules will determine the frequency at which nucleosome are ligated to Seeds. In a typical experiment a ratio of 20:1 Receptor to

Seed-Linker molecules is used. Since each mono-nucleosome has two DNA ends approximately 90% of nucleosomes will contain two Receptors, and 10% will contain 1 Receptor and 1 Seed-Linker.

Following this ligation, the chromatin is pelleted and washed several times to remove excess un-ligated Receptor to Seed-Linker molecules.

### 4: Seed Ligation.

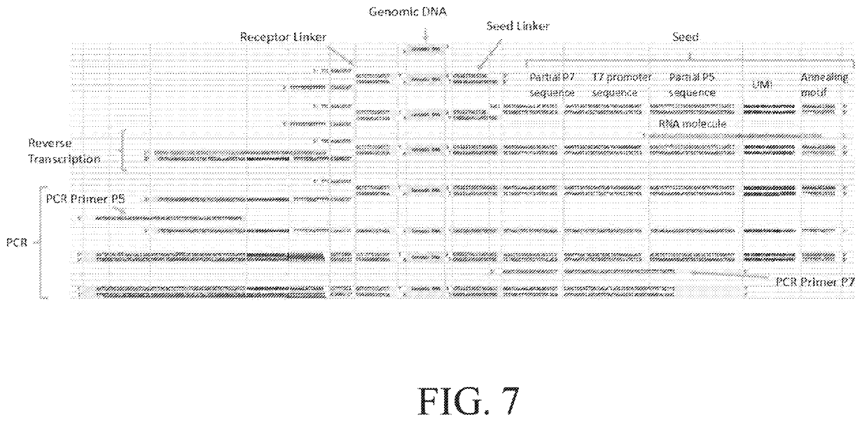

Seed nucleic acids are prepared by PCR and followed by DNA repair to remove ssDNA non-complementary to the UMI. The Seed is then digested with a specific restriction enzyme to generate a cohesive end complementary to the end of the Seed-Linker. Seed nucleic acids contain a promoter for T7 RNA polymerase, a sequence complementary to Illumina sequencing primers, a UMI and a region of homology to the Receptor DNA (FIG. 7). The purified Seed nucleic acids are then ligated to the chromatin mixture. Following this ligation, the chromatin is pelleted and washed several times to remove excess un-ligated Seed nucleic acids.

### 5: PCP Reaction.

A small quantity of the Seed Ligated chromatin is then added to a PCP reaction containing T7RNA polymerase, MMLV reverse transcriptase and appropriate reaction buffer. The reaction is incubated for 2 hours at 30° C., then 15 min at 42° C.

### 6: PCR Reaction.

Following the PCP reaction, the crosslinks are reversed, and the tagged molecules are directly amplified by PCR.

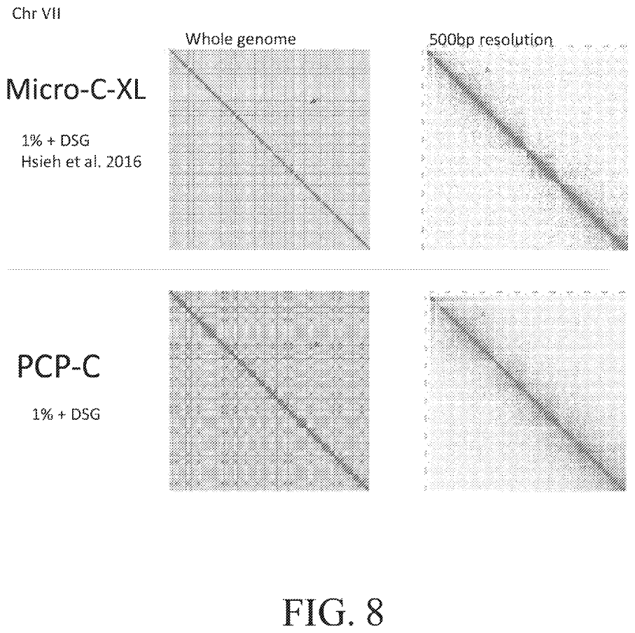

A comparison of data from PCP-C with current state of the art method (Micro-C XL) is shown in FIG. 8.

References Cited in this Example:
Multiplex chromatin interactions with single-molecule precision. Zheng M, Tian S Z, Capurso D, Kim M, Maurya R, Lee B, Piecuch E, Gong L, Zhu J J, Li Z, Wong C H, Ngan C Y, Wang P, Ruan X, Wei C L, Ruan Y. Nature. 2019 February; 566(7745):558-562. doi: 10.1038/s41586-019-0949-1. Epub 2019 Feb. 18.
Higher-Order Inter-chromosomal Hubs Shape 3D Genome Organization in the Nucleus. Quinodoz S A, Ollikainen N, Tabak B, Palla A, Schmidt J M, Detmar E, Lai M M, Shishkin A A, Bhat P, Takei Y, Trinh V, Aznauryan E, Russell P, Cheng C, Jovanovic M, Chow A, Cai L, McDonel P, Garber M, Guttman M. Cell. 2018 Jul. 26; 174(3):744-757.e24. doi: 10.1016/j.cell.2018.05.024. Epub 2018 Jun. 7.
Micro-C XL: assaying chromosome conformation from the nucleosome to the entire genome. Hsieh T S, Fudenberg G, Goloborodko A, Rando O J. Nat Methods. 2016 December; 13(12):1009-1011. doi: 10.1038/nmeth.4025. Epub 2016 Oct. 10.

### Example 2

### Proximity Copy Paste: A Methodology for Single-Molecule Analysis of Chromosome Structure

### Abstract

Genomics assays that report protein occupancy or the 3D arrangement of crosslinked and fragmented chromosomes are often used to understand how genomic information is accessed, copied, or repaired. While powerful, genomics methods only interrogate a small fraction of the available information in a sample. Important questions related to whether events are coincident or mutually exclusive; or the nature of cause-effect relationships are difficult or impossible to assay. Here, the development of new approaches to map the folding and protein occupancy of entire chromosomes is described, with single-nucleosome resolution and single molecule precision. Our method employs a proximity labelling approach that can uniquely and indelibly tag DNA, protein or other molecules that associate in 3D space. Importantly, the method can easily be tuned to different resolutions, to allow quantitative measurements of proximity over a range of distances.

### Background and Significance

A central challenge in biology is to understand the how the myriad of protein-protein and DNA-protein interactions collectively function in genome replication, maintenance, and expression. Perhaps the most used assay in chromosome biology is the Chromatin Immuno-Precipitation (ChIP) assay, which has led to a wealth of information describing the localization of proteins and their modifications across the genome. The basic ChIP approach involves crosslinking proteins and DNA together and then shearing the genome to generate distinct entities [1]. Such Crosslinked Chromatin Fragments (CCFs) are information rich: they contain not only the many proteins, RNAs and modifications that decorate to a region of the genome, but also information regarding how the genome folds in 3-D space [2]. However, when a ChIP is performed on CCFs, we interrogate a single protein; or when 3-D architecture is assayed by Hi-C, the interaction of only a subset of genomic DNA fragments is measured—meaning that the majority of the information contained within the CCF is never accessed. Simple, yet important questions of protein co-occupancy are most often inferred from independent ChIP assays leading to conclusions with questionable veracity. Adaptations like “sequential ChIP” have allowed the mapping of co-incident protein occupancy [3], but such methods are not robust, and offer little quantitative information. Beyond basic measures of co-occupancy, a neglected area of genomics is the measurement of protein/modification multimerization on chromatin. Examples include protein sumoylation [4] and post translational modification of proteins [5]. Such multimerization provides powerful regulatory roles, but there is no available assay to define when and where these events occur in the genome. ChIP and Hi-C based assays are inherently based on selecting and quantitating only positive signal, meaning that the frequency of binding events or 3D interactions cannot be directly defined or quantitated.

Assays to map higher order chromatin folding utilize a similar substrate to the ChIP assay. Hi-C and Micro-C [6-8] employ proximity ligation to join DNA fragments in close spatial relationship. Resolution of such assays is broadly dictated by the choice of nuclease and sequencing depth. Further, the “ligateablity” of DNA ends is of critical importance: only DNA termini within a certain range and having the correct 3D conformation are competent for ligation. Finally, the Hi-C—type assays only report pairwise interactions, meaning that the clustering of multi-loci in 3D space is inferred by aggregation of ensemble data. Long-read sequencing of multimer ligations can overcome some limitations in this regard [9], but ligation biases are likely.

More recently, two new methods have been developed to overcome some of the inherent limitations with Hi-C type assays. In ChIA drop, the 10X platform is used to trap an individual CCFs into droplets that are then mixed with unique DNA barcodes [10]; this method is comparatively low throughput, cumbersome and very expensive. The second approach: SPRITE, relies on split pool barcoding in which barcodes are progressively added to individual CCFs through iterative rounds of DNA ligation [11]; this method needs expensive reagents and has inherent inefficiencies. Nevertheless, because both methods introduce unique barcodes to DNA in individual CCFs they permit multi-way interactions to be mapped with single-molecule precision.

While SPRITE and ChIA drop offer a significant advance in mapping high order interactions in genomes, they are both based on the concept that a crosslinked genome must be broken into discrete entities (CCFs) by sonication before they are passed through the assay and indexed. Importantly, sonication has little to no ability to fragment chromatin into discrete units of biological significance. Thus, the act of sonication is largely fragmenting and randomizing the architecture of the genome; individual CCFs can be assayed, but at the expense of abolishing information related to the higher order connectivity and spatial arrangement between CCFs.

Assays that interrogate systems with single-molecule precision are increasingly relied upon to understand the intricacies of molecular processes hidden in ensemble assays. For example: single molecule imaging has revealed the details of the transcription cycle [12], single-molecule mapping of DNA replication by this lab has shown the remarkable processivity of the replication machinery [13]; and single molecule mapping of chromatin folding using the ChIA drop method has provided evidence for promoter looping mediated by RNA polymerase II [10].

The goal of this project is to develop new methodology to interrogate the structure of chromosomes and their protein occupancy with single molecule precision and high resolution. The method is new, conceptually simple, and can be carried out by any lab without need for expensive reagents or specialized equipment. The methodology employs a proximity tagging approach developed by this lab to map the structure of chromosomes and occupancy of proteins on DNA; importantly, spatially related molecules can be mapped without need for mechanical shearing of chromosomes, which will allow mapping 3D relationships over a vast range of distances with single molecule precision.

### Overview of the Method and Preliminary Data

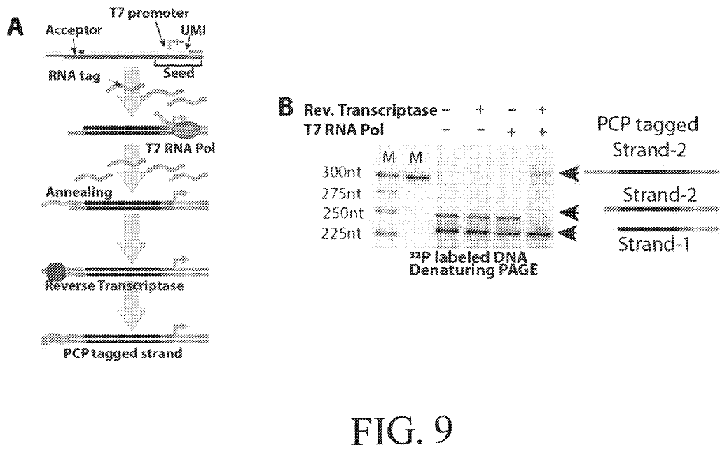

The method is based on the idea that interacting DNA molecules can be uniquely and indelibly marked by use of diffusible tags that emanate from discrete loci along chromosomes. The enzymatic basis of the tagging method is outlined in FIG. 9, panel A. The ‘seed’ contains a promoter for T7 RNA polymerase which drives transcription of an RNA tag. The tag contains a short random DNA sequence known as a Unique Molecular Identifier (UMI) and has a ‘annealing’ sequence at the 3′ end. Seeds are made using oligonucleotides encoding UMIs, ensuring that each seed nucleic acid encodes a different UMI. The ‘acceptor’ can be any DNA sequence, but the 3′ ends need to be complementary to the 3′ end annealing sequence of the RNA. The RNA that is produced by T7 transcription of the seed will anneal to the ends of nearby acceptor DNA through base pairing. Once annealed, the RNA is then a substrate for Reverse Transcriptase which will copy the RNA into DNA, thus extending the acceptor molecule with the UMI originally encoded by the seed. The use of T7 RNA polymerase to make copies of the UMI allows the synthesis of many RNA molecules from each seed.

We established model reactions to test and optimize tagging. An example reaction is shown in FIG. 9, panel B where a model substrate containing a seed and acceptor in cis is used; the full reaction—containing both T7 RNA polymerase and MMLV Reverse Transcriptase results in highly efficient and specific tagging of the target. Importantly, since RNA is made locally, acceptor molecules closest to the seed can be preferentially tagged. We term this reaction: Proximity Copy Paste or PCP as information is copied from the seed and pasted onto the acceptor—to our knowledge this combined reaction has not been previously described.

Acceptor molecules contain regions of ssDNA of specified sequence to which the tagging RNA can anneal. Molecules of interest can be engineered to contain a seed or an acceptor, but in the most basic reaction we map chromatin 3D organization by ligating seeds and acceptors into chromosomal DNA digested with a nuclease (FIG. 10). Chemical crosslinking of chromatin using a crosslinkers—formaldehyde and DSG—will ensure that 3D interactions are maintained through nuclease digestion [7]. The crosslinked and nuclease digested chromatin can then be washed, the DNA ends repaired, and acceptors plus seed nucleic acids ligated to the free DNA ends of nucleosomes. The ratio of seeds to acceptors is an important variable as it will impact the resolution of the assay: a typical reaction has 1 seed for every 10 nucleosomes, with the other nucleosome ends being occupied by acceptors. The PCP reaction is then performed on the prepared chromatin—resulting in the tagging of acceptors in proximity to the embedded seed nucleic acids (FIG. 10). Since each seed encodes a unique UMI, tagging reactions that emanated from individual seeds can be tracked by sequencing. Thus, each molecule that is tagged by a seed can be identified, which will permit single-molecule analysis of chromatin interactions. With adaptations to this method, it will be possible to determine the spatial organization of loci, chromosomes, and genomes, as well as the relative positions (and abundance) of proteins or other molecules of interest on chromatin.

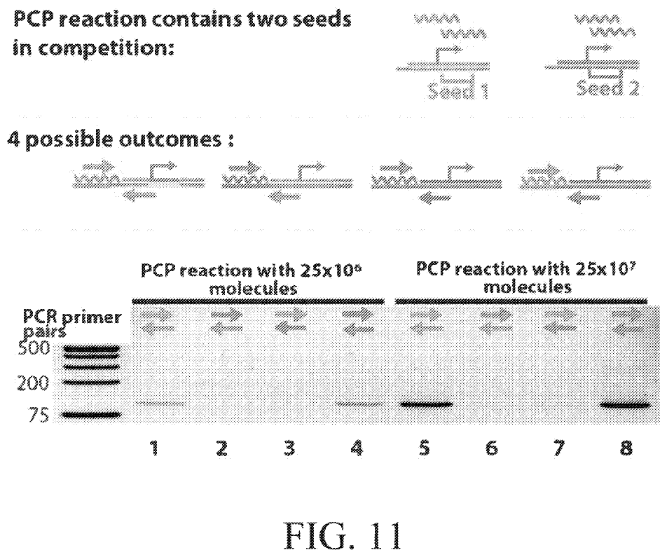

The method described herein is ultimately dependent upon the preferential tagging of acceptor molecules in proximity to the seed during PCP. Thus, we designed substrates to allow us to easily determine if the reaction preferentially occurred in cis vs trans. Using two different seed nucleic acids each with the same acceptor sequence in cis, we can use PCR to define the products of a PCP reaction when the two seeds are in competition. As shown in FIG. 11, we find that each seed in the competition reaction preferentially tags the acceptor in cis.

Next, we sought to manipulate the chromatin substrate to allow efficient nuclease fragmentation and subsequent ligation of acceptor and seed nucleic acids. Optimization of this assay is conducted in budding yeast, which is used for establishing genomics assays given the small size of the genome, ease of growth and genetic manipulation and the conserved nature of chromatin structure. The PCP can have single-molecule resolution, so it is vital that each step of the reaction is as efficient as possible and that we are able to recover the products of the PCP to generate a sequencing library. The initial step in most Hi-C type assays is the addition of formaldehyde which results in the crosslinking of proteins to DNA. Using standard conditions, we noted that the nucleosomal DNA we ultimately purified after formaldehyde crosslinking behaved very poorly in library generation—more specifically, only a sub fraction of library molecules can be amplified by PCR. We determined that the choice of quenching agent—used to remove excess formaldehyde—had substantial effect on the quality of the sequencing library, we found that use of Tris is far superior to glycine [14].

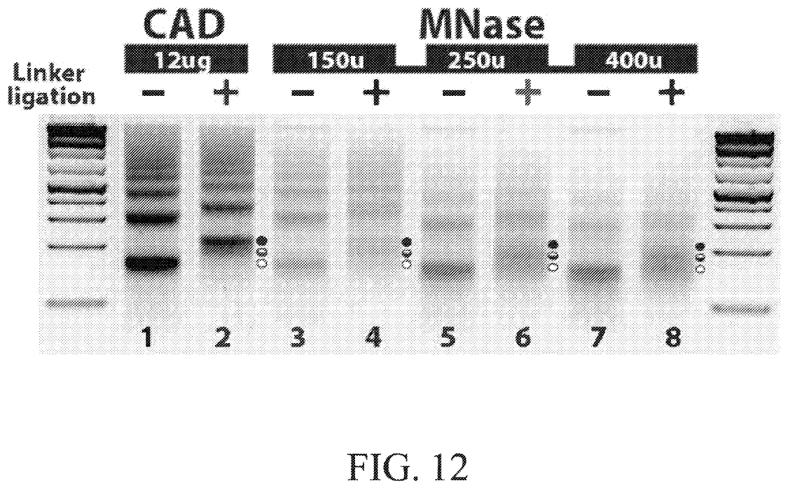

MNase is the most common nuclease used to digest chromatin into smaller regions of DNA wrapped into nucleosomes. Indeed, MNase digestion of chromatin is a key to high resolution mapping offered by the Micro-C method [8]. While the MNase has been used for the past 50 years and is very well characterized [15], digestion does require optimization to prevent cleavage within nucleosomes; in addition, the cleavage products cannot be directly ligated due to staggered cleavage sites and the presence of a 3′ phosphate. Since the PCP reaction is reliant on the ligation of DNA linkers to the ends of nuclease digested chromatin, we optimized MNase cleavage and then tested the efficiency of ligation of double strand (DS) oligonucleotides to chromatin after end-repair and A-tailing. We consistently found that we were unable to ligate DNA to the ends of a significant fraction of nucleosomal DNA (FIG. 12, note the ligation reactions are occurring on chromatin, not purified DNA), while this level of ligation is sufficient for other genomics assays, such as Micro-C, that compensate for low efficiency by increasing input, this inefficiency can significantly hamper single-molecule assays such as PCP. We noted that the efficiency of ligation diminishes as digestion proceeds, which indicated that the endo/exonucleolytic activity of MNase is digesting the nucleosomal DNA ends to such an extent that the enzymes required for end-repair and ligation can be unable to access the DNA when complexed into a nucleosome. We considered that an alternative nuclease can be better suited to our assay. Thus, we expressed and purified the Caspase Activated DNase (CAD), which has been shown to digest chromatin in a similar manner to MNase but lacks the exonuclease [16]. Use of CAD is a significant improvement over MNase allowing in near quantitative ligation of DS oligonucleotides to nucleosomal DNA (FIG. 12).

There are two single-molecule genomics assays that map high order chromatin folding: SPRITE and ChIA-Drop [10, 11]. These methods work on the principle of separating and indexing individual complexes of crosslinked chromatin using split-pool barcoding, or 10X microfluidics system. One significant advantage of PCP is that chromatin interactions can be mapped on whole chromosomes/genomes without need for physical separation of crosslinked chromatin fragments. PCP can be used on whole chromosomes/genomes provided that seeds can be evenly dispersed along the chromatin and at a controlled frequency. To achieve this, we developed a protocol to allow controlled seeding of chromatin. We synthesized two DSoligonuclotide “linker” sequences: 1, A linker with an acceptor 3′ end (acceptor-linker); 2, A linker that can ligate to a seed (seed-linker). Both linkers contain a single “T” overhang at one end, enabling them to be ligated to end-repaired and “A”-tailed CAD digested chromatin (FIG. 13). Ligation of an acceptor-linker to a nucleosome will allow that nucleosome to be tagged in a PCP reaction, whereas ligation of a seed-linker to a nucleosome will allow subsequent ligation of a seed to that nucleosome. Assuming the two types of linkers will ligate to with equal efficiency, we can control the frequency of seed ligation into chromatin by varying the ratio of the two linkers: e.g. a ratio of 1:10 (seed-linker:acceptor-linker) will ensure that approximately ⅕ nucleosomes will contain a seed (nucleosomes contain two DNA ends). Thus, the chromatin is seeded in two steps: first the two linkers are added at a fixed ratio and ligated, the chromatin is then pelleted washed to remove the excess linkers and then the seed is added with T4 DNA ligase, allowing the seed to be specifically ligated to the seed-linker. Excess seed can then be removed. The PCP reaction can then be performed on the linker and seed ligated chromatin.

The design of the seed and linker sequences has critical bearing on the efficiency of the PCP assay. The seed is synthesized by limited cycle PCR, then digested with a restriction enzyme to generate a cohesive end for ligation to the seed-linker. The acceptor-linker is designed with a long ssDNA region that is complementary to the RNA produced from the seed. The linkers are also engineered to contain sequences compatible with Illumina short-read sequencing, allowing the products of the PCP reaction to be readily amplified by PCR to generate a sequencing library that can be directly used by the Illumina machines (FIG. 13).

A key consideration when performing the PCP reaction is the number of unique seeds vs the number of sequencing reads produced from the library. At a minimum, the number of sequencing reads needs to be in excess of the number of unique seeds, however the number of molecules tagged by each seed is important when calculating sequencing depth as is the efficiency of the tagging reaction. Simple binomial probability can be used to estimate the numbers of reads required. In a typical PCP reaction, the chromatin from the equivalent of ~100,000 yeast cells (300 human cells in DNA content) is seeded at a 1:10 ratio. With current estimated PCP efficiencies of ~15%, we require more than 300 million pair end reads to approach the correct depth where each molecule is sequence multiple times. As PCP efficiencies improve through optimization, we will need to increase the number of sequencing reads or reduce the input.

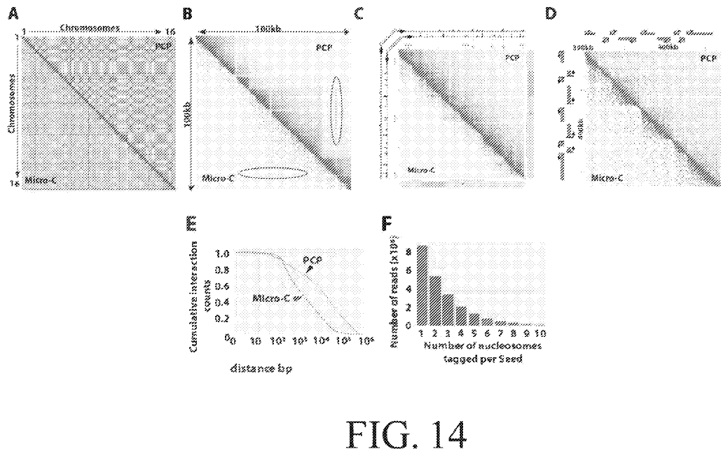

An example workflow and PCP reaction is shown in FIG. 13 the sequencing the fastq files are processed using a custom python pipeline written in the lab which maps reads to the genome and then groups sequencing reads sharing the same UMI. This basic analysis reveals that the PCP reaction on chromatin is successful and generates uniform distribution of reads across the genome. Micro-C was first developed in budding yeast in 2016; because it can provide nucleosome-level resolution, it is now becoming the “gold standard” method for revealing high resolution interaction maps in yeast, drosophila, mouse and human cells [7, 17-19]. Micro-C and PCP can have a similar maximal resolution as chromatin is digested to a similar extent with nuclease. Thus, to allow comparison, we generated a pairwise interaction matrix for the PCP data then plotted genome wide interactions for PCP vs Micro-C [20]. As shown in FIG. 14, panel A, PCP can detect 3D interactions between regions of chromatin as evidenced by the pronounced clustering of centromeres predicted due to the RABL organization of yeast chromosomes [7]. At higher resolution, we find that PCP is able to detect “TAD”-like structures in yeast chromosomes, that are not detectable by Micro-C or by Hi-C (FIG. 14, panel B). To allow further comparisons with published data, we generated a PCP map of chromatin in cells arrested in mitosis with nocodazole. Chromatin compaction during mitosis is significantly influenced by SMC complexes, Constantino et al, have used Micro-C to map SMC-mediated interactions and have shown that cohesion localizes at prominent looping sites [21]. Comparison of PCP with the Constantino dataset reveals a similar interaction map, where most points of contact are evident in both methods FIG. 14C. However, there are prominent differences: First, the PCP contact map appears to gradually decrease as distances increase, whereas the Micro-C data decays rapidly at longer distances. Second, interactions mapped by PCP that overlay with cohesin often display a “stripe” across a large region of chromatin, rather than a dot by Micro-C. Third, new interactions are revealed by PCP. Several of these appear as narrow stripes that overlay precisely with binding sites of condensin (yellow arrows FIG. 14, panel C). To our knowledge this is the first genomics assay able to map condensin function. At very high resolution in Gl cells, PCP can resolve interactions between individual nucleosomes over a range of distances not evident in Micro-C, FIG. 14, panel D. Thus, even at this early stage of optimization, PCP can capture both short- and long-range interactions with nucleosome resolution and can reveal interactions that are missed by current state of the art methods such as Micro-C. We suspect that a primary reason that PCP detects interactions missed by Micro-C is that PCP tagging is far more efficient and does not require proximity ligation and can function over a range of distances.

### Part1.1 Optimizing the Efficiency PCP.

The ability to work with a low input of chromatin is achieved through optimization of the assay. However, high resolution and true single-molecule analysis using PCP cannot be fully realized until further efficiencies are made. For example: despite seeding the chromatin at a density of ~1:10, the number of molecules tagged by each seed is centered around 1 (FIG. 14, panel F) whereas without wishing to be bound by theory each seed can tag multiple nucleosomes (e.g. ~10) at this ratio. Given that the vast majority of nucleosomes contain an acceptor (FIG. 12), the tagging reaction must be incomplete, or that other inefficiencies in library generation (e.g. PCR) can be an issue. Importantly, since the PCP reaction uses quantifiable amounts seeds and chromatin, we can optimize the reactions based on defined parameters and expectations. We detail a series of optimization experiments to improve PCP efficiency on chromatinized substrates and adaptations of the method to allow for single-molecule analysis.

To attain the highest resolution in mapping chromatin interactions, the tagging can happen rapidly—meaning RNA molecules produced from a seed can be copied into an available acceptor near to the seed. Conversely, if the tagging is inefficient, the resolution will be lessened as the RNAs will diffuse a greater distance before tagging an acceptor. The rate of RNA production by T7 RNA polymerase must be optimized with the rate of annealing and copy by Reverse Transcriptase. Annealing is influenced by the Tm of the complementary sequences; importantly, Tm is concentration dependent, so sufficient RNA needs to accumulate in proximity to the seed in order to achieve efficient tagging. PCP optimization on model templates (e.g. FIG. 9), showed that use of ideal sequences for the T7 promoter [22], improved the tagging reaction. We have also focused on optimizing the acceptor annealing sequence. We began using acceptors with short ~3 nt annealing sequences, like those used in “template switch” reactions, popular in cDNA library preparations using Reverse Transcriptase [23]. We determined that short sequences are highly suboptimal. By increasing the length of the annealing sequence, we can attain more efficient tagging; results that indicated it was important to optimize the Tm of the RNA: DNA hybrid. Ultimately, we found that a 13 nt sequence worked well.

We will undertake a more systematic analysis of which sequences function most efficiently in the PCP tagging reaction. We will specifically focus on the annealing sequences and test a range of different lengths and an assortment of different sequence compositions; care needs to be taken to limit secondary structure on the RNA tag, but this can be predicted [24]. For these reactions we will utilize model substrates that are able to tag in cis (e.g. FIG. 9) as we can rapidly synthesize them and test PCP tagging activity by gel or by PCR. We will be interested to learn how reaction temperature influences tagging with different length annealing sequences. In these experiments we will also systematically test a range of different buffers reaction temperatures and additives in promoting the efficiency of the PCP reaction. We are especially interested in the effect of crowding agents such as PEG, given their ability to limit diffusion and alter local concentration of reactants. Ultimately, it is possible that modulation of PEG levels in the PCP reaction on chromatin can significantly alter the range of tagging in the PCP.

### Part 1.2 Optimizing the Chromatin Substrate.

The biophysical properties of chromatin are can have a significant impact on the PCP reaction. Chromatin is highly charged and aggregates/precipitates readily upon addition of divalent cations [25]. Moreover, the highly basic histone tails have high affinity for nucleic acids, which can influence various aspects of PCP, including diffusion of RNA molecules in the PCP reaction. Thus, we suspect that the optimal conditions for PCP on chromatin templates can be different that those on the model substrates.

The PCP reaction on chromatin contains ~100 pg of DNA, which makes direct assessment of the PCP tagging on chromatin very challenging, however the relative efficiency can be readily assessed by the amount of product generated by a limited cycle PCR to amplify the sequencing library after the PCP reaction. Using a fixed amount of chromatin, we can test a variety of buffer conditions based on the results in Part 1.1. We will test the effect of reducing MgCl2, changing salt and crowding agents. Given the highly charged nature of chromatin, we will also test whether the addition of agents known to decondense chromatin—such as heparin, and polyglutamic acid [26]—will stimulate the PCP reaction. Finally, we will test whether the addition of detergents such as sodium deoxycholate, tween, NP40 etc, or the addition on exogenous nucleic acids to as blocking agents. Each of these conditions will also been tested on model substrates in Part 1.1, so we will have a good working knowledge as to whether the buffer conditions are compatible with PCP. Following this optimization, we will sequence a library and then test whether the results are improved by measuring the number of interactions mapped per seed at a fixed seeding ratio.

### Part 1.3. Optimizing PCP Resolution.

The extent of digestion of chromatin by nuclease, and the frequency/density that seeds are dispersed across the genome will significantly influence the resolution of the PCP reaction. Varying the extent of nuclease digestion is not a practicable method for altering the resolution in the current workflow as the library amplification method and Illumina sequencing relies on relatively short DNA fragments generated by digestion. However, altering the seed density is easily accomplished by modulating the ratio of seed:acceptor. We will experiment by using several different ratios from 1:1 to 1:100 and then perform the PCP reaction and sequence the library. The actual seed density can be deduced from the sequencing data as the seed-linkers are part of the sequencing reads, thus we can readily infer the seed density by the number of the two linkers we recover. By decreasing the seed density, without wishing to be bound by theory, the range of contact interactions will be increased, this can easily be measured by mapping pairwise interactions of molecules tagged with the same seed UMI and plotting the data as in FIG. 14, panel E.

Alterations in the seed density will also influence the rate of non-specific tagging caused by diffusion of the tagging RNAs to chromatin molecules in trans. To measure such non-specific tagging, we will prepare two different samples of chromatin that are created with either of two subtly altered acceptor-linkers (A and B) and two different types of seeds (1 and 2). The linkers and seeds will contain the same annealing sequence but can be differentiated based on variations in the sequence ligated to the nucleosome. Chromatin containing the A and B acceptor linker sequences will be seeded with seed 1 or 2 and equal quantities of the two preps of chromatin will be mixed and then used in a PCP reaction. If there is no non-specific tagging, then A and B linkers will be exclusively tagged by their seeds: 1 and 2 respectively. However, the number of times that linker A is tagged by seed 2 (and linker B tagged by seed 1) will indicate the frequency of non-specific interactions between genomes. Because the PCP reaction does not physically separate genomes, we assume that nonspecific interactions will be caused the aggregation of chromatin from different cells and chromosomes in the PCP reaction. Such aggregation can potentially be mitigated by the alteration of the PCP reaction conditions. We will test whether the increasing the PCP reaction volume and/or modulation of divalent cations, crowding agents, salt, or inclusion of detergents or chromatin decondensation agents such as polyglutamic acid, as explained herein, is beneficial. Furthermore, we will test whether inclusion of macroscopic inert particles—such as PEGylated polystyrene beads—will prevent non-specific tagging between genomes. To extend this analysis we will employ mild sonication to fragment the two genomes and then perform the PCP reaction after mixing the chromatin together. By fragmenting the genome into smaller entities, without wishing to be bound by theory, a greater degree of mixing will occur between the two differentially prepared genomes, PCP data will allow us to define how this affects the tagging reactions in cis vs trans.

### Part 1.4. Single Molecule Analysis of Chromosome Folding.

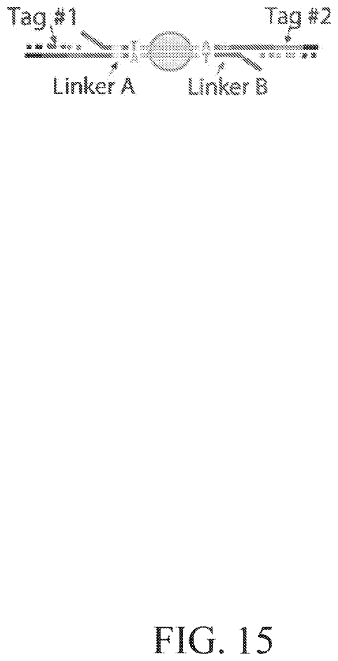

The small amount of chromatin in a PCP reaction is necessary to balance the number of unique molecules mapped to the number of sequencing reads. Given the ability to map chromatin interactions of individual nucleosomes, and the fact that PCP method does not involve mechanical shearing of the crosslinked genome, it can be possible reconstruct the spatial relationships between adjacent genomic regions tagged during the PCP reaction. Conceptually, it is possible to reconstruct the how an individual chromosome—or regions therein—fold with single molecule precision. To resolve this, we need to identify cases where each end of an individual nucleosome—occupying a defined genomic location—is tagged with two different UMIs from nearby seeds (hetero-tagged). Providing that the two UMIs can be unambiguously mapped to the same nucleosome we can infer that the seeds generating the UMIs were local to each other (see FIG. 10, panel D for illustration of this concept: nucleosomes tagged by two seeds are shown in two colors). Assuming we can achieve high PCP efficiency, this adaptation of the method can be accomplished with simple modifications to the linker sequences ligated to the nucleosomes. We will engineer new DS oligonucleotide acceptor linker sequences containing short (~4 nt) barcode sequences and ligate these to nucleosomes in the typical PCP reaction (FIG. 15). After the library is made, it is sequenced using conventional Illumina pair-end technology; this will allow the identification of which seed tagged the nucleosome, the sequence of the nucleosomal DNA as well as the barcode encoded in the linker. Importantly, if the two ends of a single nucleosome are tagged by RNAs form different seeds, each of the two UMIs from the seeds will be mapped to the same nucleosomal DNA sequence and they will have matching linker UMI sequences (one read will be reverse complement). Thus, if the linker barcodes match and the nucleosomal DNA sequence match, we can be sure that the same nucleosome was tagged by RNA from two different seeds, rather than nucleosomes from two different genomes that occupy exactly the same position.

Molecules in the sequencing library that were ligated to the seed are identifiable because the seed-linker is different to the acceptor-linker. Thus, following sequencing we can identify the genomic coordinates that each seed occupied and where the tagging RNA emanated. This information is useful in several aspects of optimization: knowing the location of the seed will allow us to map the distance from the seeds to the hetero-tagged nucleosomes. We will then use this information to extract a seed interaction matrix—containing groups of seeds linked by dual tagged nucleosomes. Each seed group will contain single-molecule information of interactions of between parts of individual chromosomes, the size and resolution of which will be defined by the number of adjacent linked seeds we obtain. The seed: linker ratio will have significant bearing on this assay, for example: low seed density will allow tagging over greater distances and more mixing of tagging RNAs from adjacent seeds. Thus, low seed density will ensure that multiple independent nucleosomes share the same tag combination, which will allow us to map interactions between seed groups with greater confidence.

### Part 1: Outcomes and Alternative Approaches.

The PCP assay is fundamentally different to Hi/Micro-C, SPRITE and ChIA-drop in that we can accurately predict and measure the efficiency of the tagging reaction on chromatin. This allows us to systematically test a variety of reaction conditions to identify optimal parameters. In addition, ability to seed the reaction at different densities offers the ability to fine tune the reaction to different resolutions and to detect interactions over longer distances suiting specific biological questions. We have experience with the PCP assay as it has taken a few years to achieve a workable assay; we are confident that improvements can still be made. The maximum theoretical efficiency of PCP on chromatin will not be achieved but improving on the ~10% tagging efficiency we currently have is certainly achievable. Optimization will take time as the full validation of each improvement requires in-depth sequencing and analysis before conclusions can be drawn and new experiments planned. Other issues we can encounter relate to the effects that variations in chromatin structure have on the PCP assay; for example, it is known that biophysical properties of chromatin is altered through histone acetylation, such changes can influence PCP efficiency and thus give rise to local variations in results that confounds straightforward analysis. Hand in hand with the method development is the development and implementation of computational methods to interpret the data. The pipelines and analysis has been accomplished in this lab and we are confident that we have the necessary skills and collaborators to complete this work. While the workflow of PCP is fundamentally different to Hi-C type assays, the literature and methods applied to Hi-C data for normalization, visualization and analysis can be easily implemented to analyze the bulk PCP data. The single-molecule analysis is certainly more complex, but our pipelines need minor modifications to handle this data. In addition, new tools are available to work with single-molecule chromatin interaction data (ref). The results of the PCP assay will also need to be validated to confirm that the interactions we detect are real. Primarily, this can be accomplished by comparison with published datasets conducted by conventional assays (e.g. FIG. 14). However, our preliminary data shows that PCP can detect longer range interactions that have not been reported e.g., TADs. Validation of such interactions is more difficult if orthogonal assays cannot detect them, but genetic manipulation of yeast will allow us to perturb proteins involved in these interactions (e.g. SMC complexes) or make specific deletions of loci that appear to function at the boundary of the TADs (which are highly expressed genes) and test how this affects the PCP readout.

### Part 2. Protein Detection by PCP

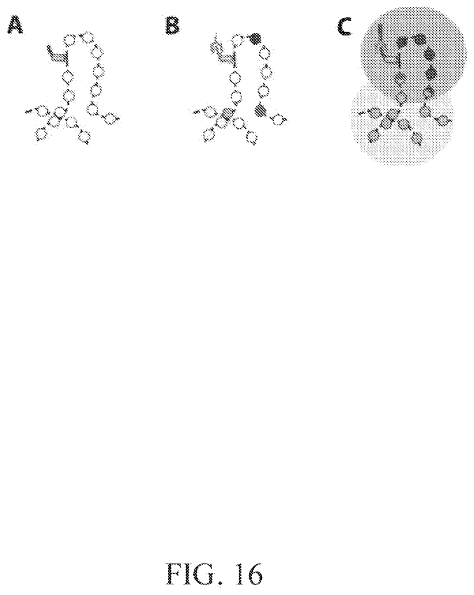

A comprehensive understanding of chromatin organization and the occupancy of various DNA binding proteins are gained by performing independent assays such as ChIP and Hi-C and cross-referencing the data. As such, correlation is often used to assert co-occupancy, which can, or cannot, be true. In this section, we detail modifications to the PCP reaction that can allow the simultaneous mapping of protein localization and genome folding. In principle, the PCP reaction can tag any molecule containing an acceptor sequence that is in proximity to the seed. In the basic method, acceptors are ligated to digested chromatin, allowing the spatial relationship between nucleosomes to be deduced. Besides nucleosomes, the chemical crosslinking used to generate the substrate for the PCP reaction will also trap myriad of other proteins and RNA on the chromatin template. The presence of proteins on the genome can theoretically be mapped by PCP if an antibody, or other specific affinity reagent for a target protein is functionalized with an oligo acceptor sequence. In this iteration of the method, a functionalized antibody is added to the standard PCP rection as described in Part 1. Tagging RNAs produced from seeds ligated to the chromatin would tag nucleosomes and functionalized antibodies in proximity to each seed (FIG. 16). Following sequencing, the tagged antibody sequences can be identified and associated with specific loci according to the identity of the nucleosomes that share the same UMI from the same seed. Thus, with simple modifications, the PCP reaction can map the general positions of proteins in the 3D genome. Importantly, numerous proteins can be simultaneously mapped—and counted—provided that distinct affinity reagents are available. Hereafter, the method to map protein localization will be termed PCP-ID.

While functionalization of antibodies with oligonucleotides has become common practice [27], our trial experiments showed variable levels of efficiency of coupling DNA to primary antibodies. We reasoned that nanobodies—single domain camelid antibodies—can provide a better suite of reagents to allow us to optimize the method. Nanobodies can be made recombinantly and can target specific epitopes such as the ALFA-tag [28] and different isotypes of IgG with extremely high efficiency [29]. Thus, nanobodies can be used against specific tags engineered into proteins; or against a vast array of primary antibodies, providing the isotype is compatible. Furthermore, nanobodies can be functionalized in highly specific ways and purified to near homogeneity.

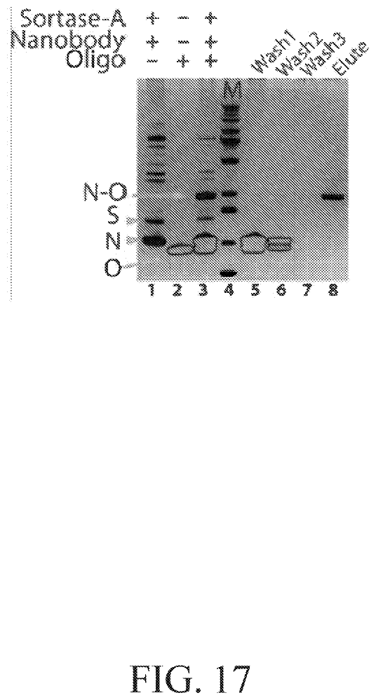

The strategy for functionalizing nanobodies relies on two proteins: a nanobody and Sortase-A each of which we express in e. coli and purify in the lab. Sortase-A is used to simultaneously cleave a specific sequence at the C-terminus of the nanobody an functionalize the nanobody with DBCO [30] [31]. An oligonucleotide with a 5′ azide, is then reacted with the DBCO on the nanobody in a “click” reaction to efficiency append an oligo to the nanobody. Optimization now allows this lab to purify large quantities of pure nanobodies functionalized with specific oligonucleotide sequences (FIG. 17).

### Part 2.1 Nanobody Detection by PCP.

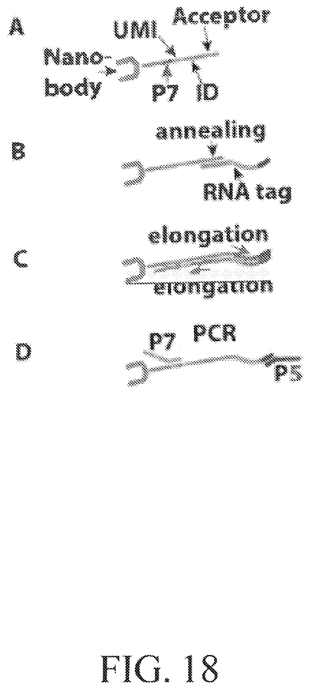

We have designed the oligonucleotide to attach to the nanobody to contain sequences compatible with the standard PCP reaction and subsequent library generation. The sequence contains an annealing sequence to allow tagging, an ID sequence to allow the identification of the nanobody and a short UMI will allow accurate quantitation of the number of different nanobodies tagged by a seed after library sequencing (FIG. 18).

The PCP-ID reaction will utilize the same crosslinked, CAD digested chromatin substrate as the PCP, explained herein. The chromatin substrate will be seeded at high density to allow high-resolution mapping of nanobody locations. We will use an engineered yeast strain containing an ALFA-tagged Rap1 protein, which will serve as a control to optimize the assay. Rap1 is a general regulatory transcription factor, whose binding to the genome has been characterized [32]. Prior to the PCP reaction, the anti-ALFA nanobody will be added to the chromatin, but great care needs to be taken to ensure that the unbound nanobody is removed. Unlike a ChIP assay, PCP-ID does not immobilize chromatin on beads as we found that chromatin tends to bind non-specifically to any affinity matrix. However, since the crosslinked, PCP digested chromatin readily pellets upon centrifugation in buffers containing divalent cations, low-speed spin in a centrifuge are sufficient to allow buffer exchange and removal of smaller molecules, such as enzymes, oligonucleotides and presumably, nanobodies.

To optimize the wash conditions, we will utilize two yeast trains: a WT and the Rap1-ALFA tagged strain. Chromatin will be prepared in the standard manner for the PCP assay and then the ALFA-nanobody functionalized with an oligonucleotide will be added to each of the chromatin reactions. We will then trial different centrifugation and wash conditions, varying buffer, salt, detergents, and other components to remove the nanobody from the control and retain it in the Rap1-ALFA chromatin. The presence of the nanobody on chromatin can be assessed by PCR to detect the oligonucleotide attached to the nanobody. After appropriate wash conditions are found, we will then proceed to the PCP-ID reaction using the ALFA-nanobody with acceptor oligonucleotide attached.

We will modify the analysis pipeline to accommodate the extra information provided by the oligonucleotide sequence attached to the nanobody. Given that the genomic location of the nanobody is not directly coded in the tagging reaction, we must infer the position of the nanobody from the locations of nucleosomes tagged by the same seed. Next, we will test whether two different nanobodies can be detected in the same PCP-ID reaction. Using the same strategy listed for the ALFA-tag, we will create a SPOT-tagged strain of the Abf1 protein. SPOT is another epitope-tag recognized by a specific nanobody [33]; Abf1 is well characterized transcription factor [32]. We will purify the SPOT nanobody and functionalize with an oligonucleotide with a different “ID” sequence. We will then confirm that the SPOT nanobody is able to map the locations of Abf1 in the genome[32]. Following this we will test whether addition of both nanobodies to a PCP-ID reaction allows the mapping of the occupancy of both molecules simultaneously.

The inclusion of a UMI in the nanobody oligonucleotide potentially allows the number of nanobodies tagged by an individual seed to be directly counted. In this case, if two different nanobodies are tagged by the same seed, they will share the same UMI from the seed, however, these two independent tagging events can be distinguished as the short UMI encoded in the nanobody oligonucleotide will be different. Thus, the number of tagging events can be directly determined and quantitated, potentially allowing protein occupancy to be defined. Such an approach is dependent upon efficient tagging, but this can be tested using a strategy in which target proteins contain multiple copies of the same epitope tag. Thus, we will recombinantly express and purify a Rap1 protein that contains 1 or 3 copies of the ALFA tag fused in tandem (with short spacers to allow simultaneous binding of the ALFA nanobody). We will then perform in vitro binding experiments (e.g. band shifts) to test that multiple nanobodies can indeed bind in the predicted manner. Next, we will engineer yeast strains to contain Rap1 with 1 or 3 copies of the ALFA-tag. We will then use these strains in the PCP-ID reaction to examine whether we are able to map the correct number of ALFA nanobodies to the correct strain. The strains with 3 ALFA-tags fused to each Rap1 protein will serve as important tools for optimization of the PCP-ID assay, this is because (without wishing to be bound by theory) a known number of nanobodies per molecule of Rap1 can be attained. If we fail to achieve the expectation, i.e., 1 nanobody instead of 3, this will indicate that Rap1 is bound near the seed, but that the tagging reaction or another aspect of the library generation procedure is inefficient. We will then use these reagents to optimize nanobody binding and wash conditions and PCP reaction to allow us to test whether we can capture the targeted number of nanobodies per Rap1.

### Part 2.2 Antibody Detection by PCP.

The ability to utilize primary antibodies in the PCP-ID reaction can significantly broaden the utility of the assay. While it is possible to attach oligonucleotides directly to antibodies, we will focus optimization using “secondary” nanobodies, which bind primary antibodies of a specific isotype [29]. In this approach, a primary antibody will be added and allowed to bind to its target in chromatin. A secondary nanobody—functionalized with an acceptor oligonucleotide—will then be introduced to bind to the primary antibody. The unbound antibody and nanobodies will be removed and the PCP reaction will take place to tag the nanobody, allowing its position abundance and identity to be deduced using the principles discussed above.

To optimize the use of antibodies we will again make use of affinity tags engineered on proteins of interest. The Flag-tag is widely used in ChIP assays; quality antibodies are available which give rise to high quality data. We will engineer the Rap1 transcription factor to contain a Flag-tag and also purify a secondary nanobody that is specific for the M2 antibody. The PCP-ID assay will be conducted as before except with the inclusion of the primary antibody. Unbound antibody and nanobodies will be removed before the PCP reaction takes place. Some optimization will be needed to ensure primary antibodies bind efficiently to the chromatin and that unbound antibody is efficiently removed. One strategy to rapidly accomplish this optimization is to perform immunoblotting of chromatin using a secondary—HRP conjugated—antibody to monitor the presence of the primary antibody on the chromatin. In this case, a negative control strain without a Flag-tagged Rap1 will be used to monitor the efficiency of washes. Using the Rap1 protein as a target for the optimization will allow us to compare between the direct nanobody and primary antibody strategies for performing PCP-ID. Successful completion will allow us to move to using two antibodies simultaneously.

Part 2.3 Seeding the PCP with Nanobodies.

The use of an acceptor on a nanobody as described herein, can allow the mapping of protein occupancy whilst also mapping genome-wide 3D chromatin contacts. However, this approach requires a large number of sequencing reads as it essentially interrogates 3D contacts within a genome. A more targeted approach—somewhat analogous to Hi-ChIP [34]—would be to attach a seed to a nanobody and then perform the PCP reaction to map the localization and 3D interactions of the protein of interest. In this case, a seed attached to the nanobody will produce the tagging RNA and then any nucleosome in proximity (that contains an acceptor) will be tagged. To optimize this assay, we will develop strategies of functionalizing a nanobody with a seed. This can be relatively straightforward given our preliminary data (FIG. 17). For trial experiments we will make use of the ALFA-tagged Rap1 strain and an anti-ALFA nanobody for seed functionalization. Next, we will test whether the nanobody-seed fusion can specifically bind chromatin, and how effectively unbound nanobody can be removed. Finally, we will prepare chromatin and only ligate acceptor-linkers, we will then bind the nanobody seed and test whether a PCP reaction can map the binding locations of the Rap1 protein. The time of PCP reaction will have a significant bearing on this version of the assay as the RNA will progressively diffuse from the seed and tag acceptors at increasing distance over time.

### Part 2. Outcomes and Alternative Approaches.

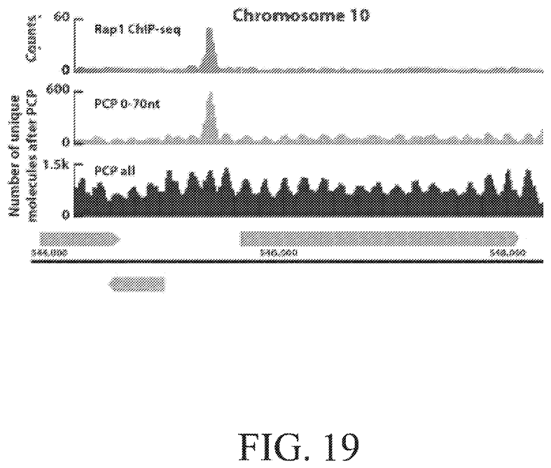

The ability to map protein localization within the context of 3D chromosome organization will allow significant new insight into various aspects of genome biology. Given that the PCP reaction has already demonstrated relatively efficient tagging on chromatin templates, we are confident that the PCP-ID approach will be successful. Nevertheless, a range of problems must be overcome to develop a useful assay. First, proteins being investigated (e.g. Rap1) need to be captured on the chromatin being assayed by PCP-ID. We are confident that this is the case as we find that the DNA bound (and protected from CAD digestion) by Rap1 is tagged in the standard PCP assay (FIG. 19). Second, we need to achieve specific and high occupancy of the nanobody on its targets. This is possible given the affinity of the ALFA and Spot tag systems, but the use of primary antibodies in Part 2.2 can prove more difficult to achieve as purified antibodies often contain aggregates that can interfere with various steps of the assay. Third, non-specific binding must be carefully controlled with appropriate use of wash buffers. The DNA attached to the nanobody can promote non-specific binding to chromatin, and we can include single-strand binding proteins to dimmish this. Fortunately, given the wealth of ChIP-seq data to serve as comparison, we can rapidly assess whether the assay is working as intended. Fourth, the crosslinking reagents can alter or mask the epitope for the nanobody/antibody, this is less of a concern with the ALFA-tag as it does not contain lysine. However, careful optimization of the crosslinking conditions can be required for other epitopes. Importantly, the PCP-ID assay will provide quantitative information of protein occupancy on the genome; this is because both the number of binding events (numerator) and the total number of binding sites (denominator) are measured in the same assay. It will also be possible to extract single-molecule information from the PCP-ID data, allowing interactions involving single proteins can be obtained and assessed independent to the whole dataset. Thus, the binding and 3D contact landscape of an individual protein can be directly extracted from the data and analyzed.

### Part 3 PCP in Mammalian Genomes.

A limitation in use of PCP in mammalian cells is the need for large numbers of sequencing reads to achieve single-molecule resolution. However, with the cost of sequencing already less than $1 per million reads, and predicted to decrease, the routine analysis of large genomes with PCP will become feasible in the coming years. Notably, even with sequence coverage that doesn't capture the complete diversity in the library, the PCP assay can still map complex interactions at high resolution. In this section, we will optimize the PCP assay for use in Mouse ES cells[35]. These cells have recently been used in Micro-C and there are published datasets for the comparison and validation of PCP [17]. The majority of the PCP reaction conditions we have optimized in yeast can be directly transferred to metazoan cells—in fact, preparation of mammalian chromatin is significantly less complex than yeast due to the lack of a cell wall. Optimization will be needed, but, without wishing to be bound by theory few difficulties can occur as crosslinking conditions have been optimized for Micro-C; and CAD, is, by nature, well suited to digest chromatin in metazoan cells[36]. We will trial PCP reactions by comparing the library yield with different amounts of input chromatin. Once satisfactory conditions are found we will test how altering the seed density in a fixed amount of chromatin affects the resolution and readout of the PCP assay. While it is currently impracticable to sequence at sufficient depth to map all interactions, we will initially aim for high read number, with this data we will compare with published Micro-C data to test the relative performance of PCP. We will then down-sample the data to find a minimal appropriate read number for further optimization experiments. The relative efficiency of the PCP reaction will have significant impact on quantities of cells and sequencing reads: lower PCP efficiency will lower the diversity of the library, and so will dictate the use for more input cells, or a fewer sequencing reads to attain coverage of interest. Large metazoan genomes, whose TAD and sub-TAD structures form over very large genomic distances, will require a different seed ratio than is optimal in yeast. Once the ideal conditions are obtained, we will progressively decrease the amount of chromatin and test whether we can obtain high coverage of interactions needed for single-molecule analysis. For example, a single human cell will contain ~30 million nucleosomes, 5x coverage can require ~150 million reads; following this logic, minimal coverage of a PCP library from 50 cells can be achieved with ~7 billion reads. However, the efficiency of the PCP reaction will significantly affect the number of sequencing reads needed to sample the full diversity of the library, thus we will modulate the input amount and sequencing depth accordingly.

### Part 3. Outcomes and Alternative Approaches.

Without wishing to be bound by theory, PCP will be easily adapted to metazoan cells, some issues related to differential tagging or CAD digestion of euchromatin vs heterochromatin can arise, but such deficiencies can be handled with more optimization. Adequate sequence coverage is a clear impediment to full implementation of the PCP assay in large genomes, but provided the reactions are seeded correctly and the input is low, 3D contacts can easily be mapped with read numbers comparable to Micro-C. With low input and single molecule readouts, careful selection of a homogeneous population needs to be accomplished in order to achieve consistent results.

### Conclusion

Our understanding of how biological systems operate is fundamentally influenced by the type of assays we employ. The approaches outlined in this proposal will offer a new suite of tools that can be used to address an array of significant questions for which we have limited insight. PCP offers a highly quantitative, tunable, assay that can map chromatin interactions over long-ranges, whilst also mapping protein occupancy. Beyond the methods discussed in this proposal, the PCP method can be adapted to study other aspects of biology where proximity mapping is needed. Most immediately, the relative locations of RNA molecules can be mapped with simple strategies to ligate acceptors onto the ends of RNA. Yet, the ability to tag over longer distances and attach acceptors or seeds to affinity reagents such as nanobodies will permit the relative 3D mapping of a range of proteins and organelles that don't necessarily directly interact with DNA. Assuming crosslinking preserves cellular structures, the principles of mapping the 3D genome developed in this proposal will be applicable to myriad of biological questions.

References Cited in this Example:
1. Milne, T. A., K. Zhao, and J. L. Hess, Chromatin immunoprecipitation (ChIP) for analysis of histone modifications and chromatin-associated proteins. Methods Mol Biol, 2009. 538: p. 409-23.
2. Lieberman-Aiden, E., et al., Comprehensive mapping of long-range interactions reveals folding principles of the human genome. Science, 2009. 326(5950): p. 289-93.
3. Beischlag, T. V., G. G. Prefontaine, and O. Hankinson, ChIP-re-ChIP: Co-occupancy Analysis by Sequential Chromatin Immunoprecipitation. Methods Mol Biol, 2018. 1689: p. 103-112.
4. Flotho, A. and F. Melchior, Sumoylation: a regulatory protein modification in health and disease. Annu Rev Biochem, 2013. 82: p. 357-85.
5. Hsin, J. P. and J. L. Manley, The RNA polymerase II CTD coordinates transcription and RNA processing. Genes Dev, 2012. 26(19): p. 2119-37.
6. Krietenstein, N. and O. J. Rando, Mammalian Micro-C-XL. Methods Mol Biol, 2022. 2458: p. 321-332.
7. Hsieh, T. S., et al., Micro-C XL: assaying chromosome conformation from the nucleosome to the entire genome. Nat Methods, 2016. 13(12): p. 1009-1011.
8. Hsieh, T. H., et al., Mapping Nucleosome Resolution Chromosome Folding in Yeast by Micro-C. Cell, 2015. 162(1): p. 108-19.
9. Li, Z., et al., Pore-C simultaneously captures genome-wide multi-way chromatin interaction and associated DNA methylation status in Arabidopsis. Plant Biotechnol J, 2022. 20(6): p. 1009-1011.
10. Zheng, M., et al., Multiplex chromatin interactions with single-molecule precision. Nature, 2019. 566(7745): p. 558-562.
11. Quinodoz, S. A., et al., Higher-Order Inter-chromosomal Hubs Shape 3D Genome Organization in the Nucleus. Cell, 2018. 174(3): p. 744-757 e24.
12. Li, J., et al., Single-Molecule Nanoscopy Elucidates RNA Polymerase II Transcription at Single Genes in Live Cells. Cell, 2019. 178(2): p. 491-506 e28.
13. Claussin, C., J. Vazquez, and I. Whitehouse, Single-molecule mapping of replisome progression. Mol Cell, 2022. 82(7): p. 1372-1382 e4.
14. de Jonge, W. J., et al., An Optimized Chromatin Immunoprecipitation Protocol for Quantification of Protein-DNA Interactions. STAR Protoc, 2020. 1(1): p. 100020.
15. Noll, M., Subunit structure of chromatin. Nature, 1974. 251(5472): p. 249-51.
16. Allan, J., et al., Micrococcal nuclease does not substantially bias nucleosome mapping. J Mol Biol, 2012. 417(3): p. 152-64.
17. Hsieh, T. S., et al., Resolving the 3D Landscape of Transcription-Linked Mammalian Chromatin Folding. Mol Cell, 2020. 78(3): p. 539-553 e8.
18. Levo, M., et al., Transcriptional coupling of distant regulatory genes in living embryos. Nature, 2022. 605(7911): p. 754-760.
19. Krietenstein, N., et al., Ultrastructural Details of Mammalian Chromosome Architecture. Mol Cell, 2020. 78(3): p. 554-565 e7.
20. Durand, N. C., et al., Juicebox Provides a Visualization System for Hi-C Contact Maps with Unlimited Zoom. Cell Syst, 2016. 3(1): p. 99-101.
21. Costantino, L., et al., Cohesin residency determines chromatin loop patterns. Elife, 2020. 9.
22. Conrad, T., et al., Maximizing transcription of nucleic acids with efficient T7 promoters. Commun Biol, 2020. 3(1): p. 439.
23. Zhu, Y. Y., et al., Reverse transcriptase template switching: a SMART approach for full-length cDNA library construction. Biotechniques, 2001. 30(4): p. 892-7.
24. Zuker, M., Mfold web server for nucleic acid folding and hybridization prediction. Nucleic Acids Res, 2003. 31(13): p. 3406-15.
25. Clark, D. J. and T. Kimura, Electrostatic mechanism of chromatin folding. J Mol Biol, 1990. 211(4): p. 883-96.
26. Villeponteau, B., Heparin increases chromatin accessibility by binding the trypsin-sensitive basic residues in histones. Biochem J, 1992. 288(Pt 3)(Pt 3): p. 953-8.
27. Wiener, J., et al., Preparation of single- and double-oligonucleotide antibody conjugates and their application for protein analytics. Sci Rep, 2020. 10(1): p. 1457.
28. Gotzke, H., et al., The ALFA-tag is a highly versatile tool for nanobody-based bioscience applications. Nat Commun, 2019. 10(1): p. 4403.
29. Pleiner, T., M. Bates, and D. Gorlich, A toolbox of anti-mouse and anti-rabbit IgG secondary nanobodies. J Cell Biol, 2018. 217(3): p. 1143-1154.
30. Fabricius, V., et al., Rapid and efficient C-terminal labeling of nanobodies for DNA-PAINT. Journal of Physics DApplied Physics, 2018. 51(47).
31. Massa, S., et al., Sortase A-mediated site-specific labeling of camelid single-domain antibody-fragments: a versatile strategy for multiple molecular imaging modalities. Contrast Media Mol Imaging, 2016. 11(5): p. 328-339.
32. Gutin, J., et al., Fine-Resolution Mapping of TF Binding and Chromatin Interactions. Cell Rep, 2018. 22(10): p. 2797-2807.
33. Braun, M. B., et al., Peptides in headlock—a novel high-affinity and versatile peptide-binding nanobody for proteomics and microscopy. Sci Rep, 2016. 6: p. 19211.
34. Mumbach, M. R., et al., HiChIP: efficient and sensitive analysis of protein-directed genome architecture. Nat Methods, 2016. 13(11): p. 919-922.
35. Pettitt, S. J., et al., Agouti C57BL/6N embryonic stem cells for mouse genetic resources. Nat Methods, 2009. 6(7): p. 493-5.
36. Widlak, P. and W. T. Garrard, Unique features of the apoptotic endonuclease DFF40/CAD relative to micrococcal nuclease as a structural probe for chromatin. Biochemistry and Cell Biology, 2006. 84(4): p. 405-410.

### Example 3

Understanding the principles of three-dimensional genome organization is essential to comprehend genome maintenance, expression, and duplication. Current Hi-C based methods use fragmentation of crosslinked chromatin followed by proximity ligation to detect DNA molecules whose extremities can be ligated together. While powerful, these approaches are limited because only ligatable, pairwise interactions are mapped, and the assay is difficult to quantitate as the prevalence of an interaction over the population cannot be assessed. Single-molecule methods such as ChIA-drop and SPRITE overcome the need for proximity ligation by physically isolating and uniquely barcoding crosslinked chromatin fragments. While these methods allow multiway interactions, they are costly, have relatively low throughput and arbitrarily shear the genome.

We developed a new technology to map 3D genomic organization. Our method—called Proximity Copy Pasting (PCP)—is based on a new reaction that uses locally produced diffusible nucleic-acid barcodes to tag molecules in proximity. The tagging reaction labels multiple DNA extremities in proximity and is not hindered by proximity ligation. PCP generates a single-molecule readout without need for physical separation of molecules to be tagged, thus spatial proximity can be mapped in complex mixtures of chromatin. Together, PCP has several advantages over currently available methods while being more economic, easier to implement and higher throughput.

Using budding yeast as a model organism, we recapitulate known features of genome organization such as centromere clustering, MAT locus interactions, small scale chromatin interaction domains (CID). We also detected new features: larger scale TAD like structures, highly expressed genes and tRNA ‘stripes’ of interactions. Furthermore, PCP generates sufficient resolution to map the position and interactions of individual nucleosomes and transcription factors. Importantly, the resolution of PCP can be tailored to detect interactions over different scales and is therefore able to map proximity over distances that are beyond the reach of proximity ligation.

### Example 4

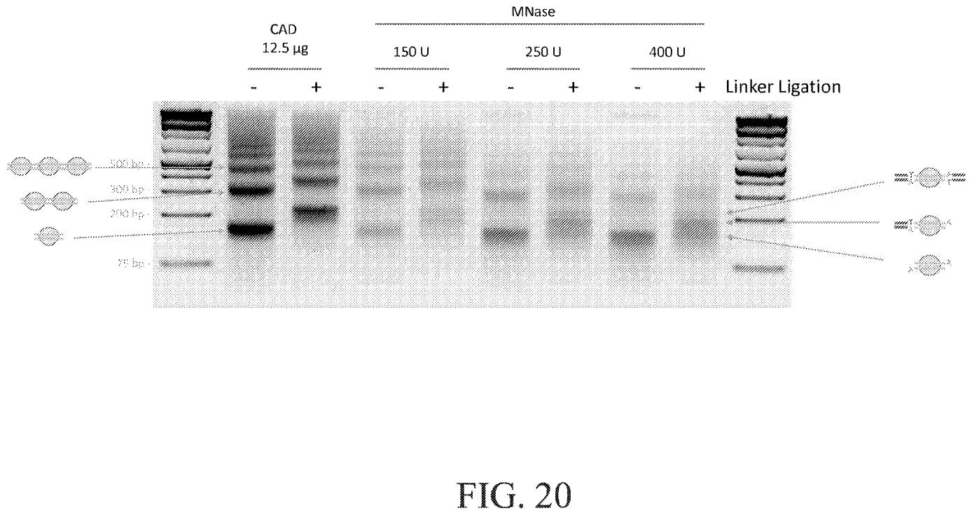

Referring to FIG. 20, CAD chromatin digestion allows efficient linker ligation. Despite similar chromatin digestion at 150U of MNase compared to CAD 12.5 μg, the ligation of linker is more efficient on CAD digested chromatin. Digestion of chromatin with higher amounts of MNase leads to chromatin over-digestion and decrease in linker ligation efficiency.

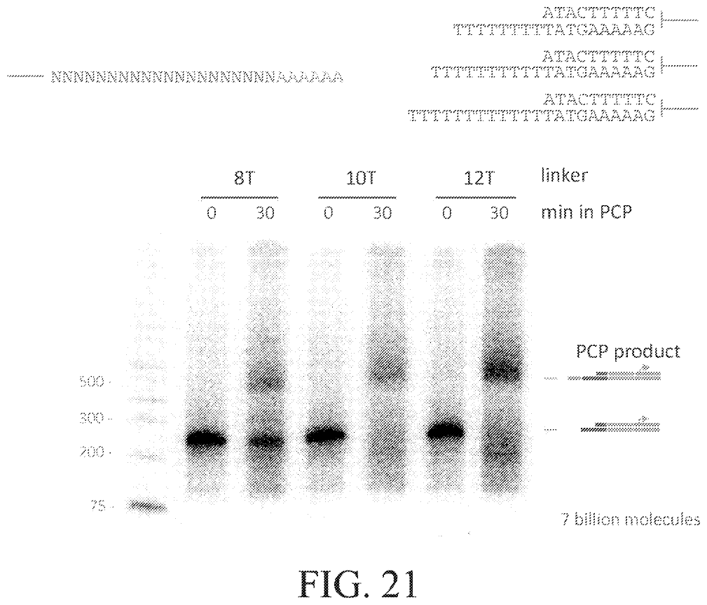

Referring to FIG. 21, longer T-tails reach higher efficiency of the PCP reaction.

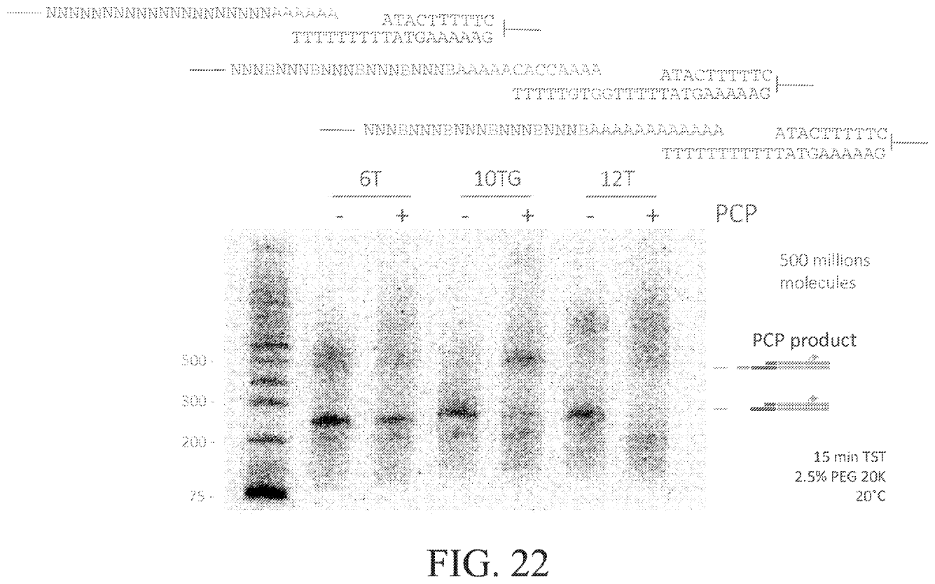

Referring to FIG. 22, the 10TG motif provides efficiency with a discrete size product. The homopolymeric 12T motif results in high PCP efficiency but the product is less discrete.

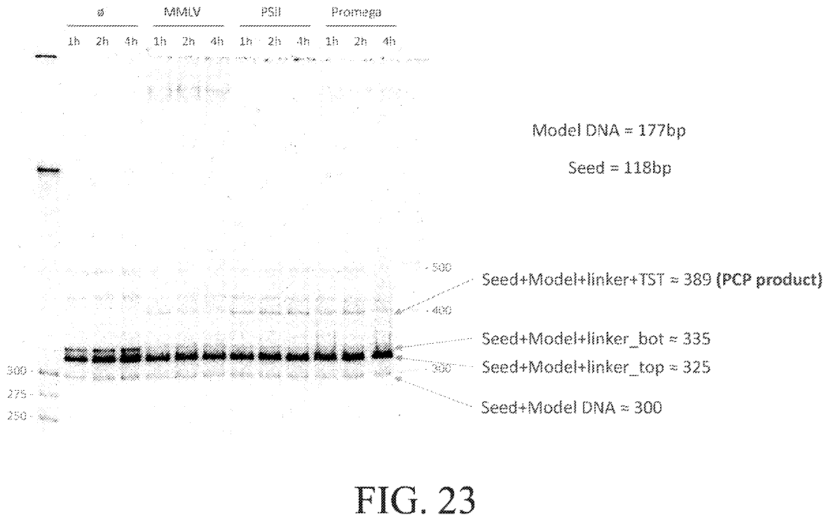

Referring to FIG. 23, ProtoScript II (NEB M0368) gives a distinct PCP that is stable over long reaction times.

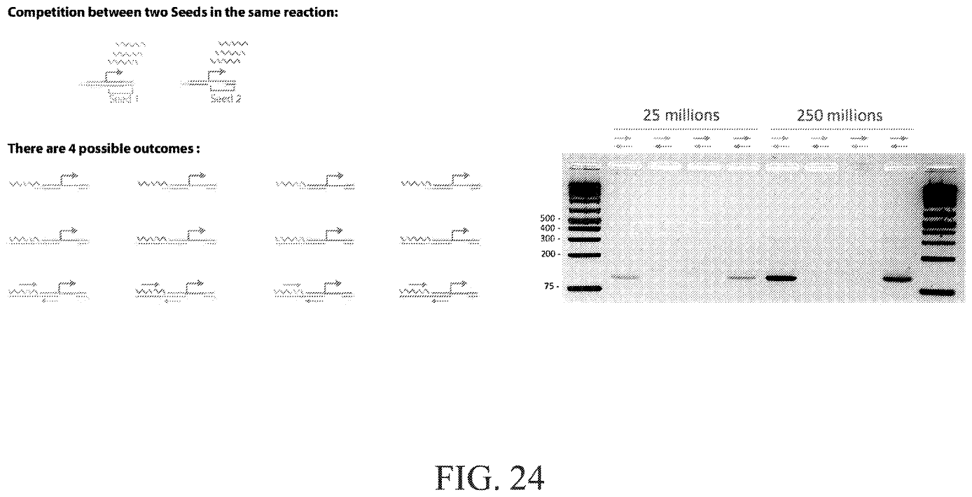

Referring to FIG. 24, experiments were performed to test if molecules in proximity are preferentially tagged. Two Seeds were introduced into the same reaction. Each seed produced a slightly different RNA that can tag the receptor on either seed. Whether a Seed tags in cis or in trans can be determined by PCR. The results show that Seeds preferentially tag a molecule in cis.

Referring to FIG. 25, decay of the frequency of interaction relative to the genomic distance for PCP and Micro-C is shown. Altering the Seed to Receptor ratio alters the resolution of the PCP assay. Reducing the relative amount of Seeds in the reaction, from ⅕ (blue) to 1/20 (orange), increases the mapped contact distances.

### Example 5

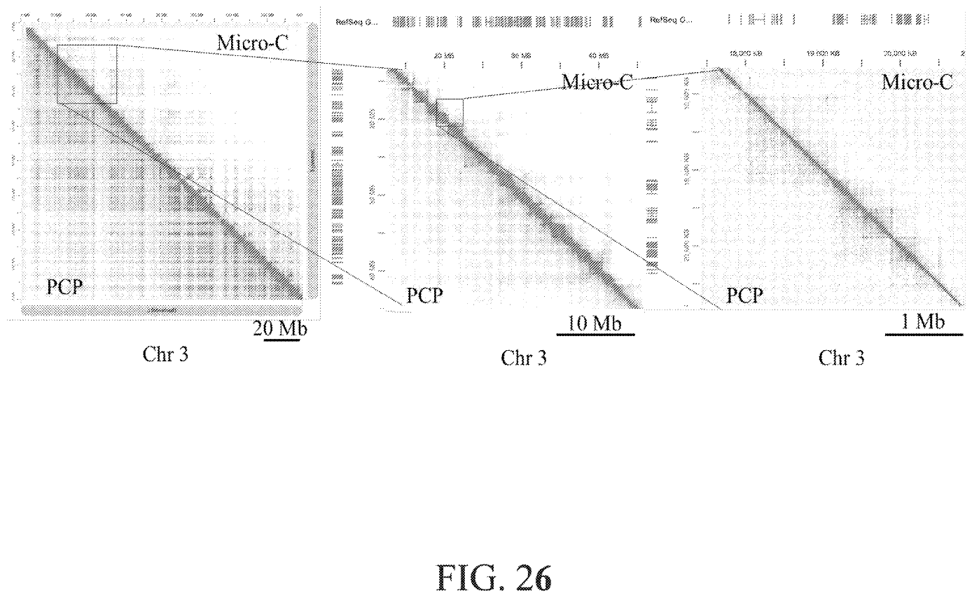

Referring to FIG. 26, PCP recapitulates the 3D features detected by Micro-C at several scales as viewed with Juicebox.

Referring to FIG. 27, the DNA substrate (Seed DNA) contains a single binding site (Rap1BS) for the yeast Rap1 transcription factor, a ‘Seed’ and a receptor (Panel A). Three different Rap1 proteins are prepared with differing numbers of copies of the ALFA epitope tag: Rap1_notag; Rap1_1xALFA; Rap1_3xALFA (Panel B). The ALFA tag is detected with a nanobody with a single-strand oligonucleotide receptor covalently attached (Panel C). Three separate reactions are prepared, each containing the same quantity of Seed DNA substrate, the same quantity of anti-ALFA Nanobody and the same quantity of one of the three Rap1 proteins (Panel D). Competitor oligonucleotides are added into each reaction the prevent tagging in trans (FIG. 28). In each reaction the Rap1 protein will bind to the substrate DNA, and the nanobodies will bind to the ALFA epitope tags, if present (Panel D). After PCP is conducted on each reaction, the amount of tagged nanobodies will reflect the number of Rap1 epitopes bound by the nanobody in vicinity of the seed, thus a greatest proportion of nanobodies will be tagged in the reaction containing the Rap1_3ALFA protein. The Seed DNA substrate will be tagged to the same extent in each of the reactions, serving as a control for PCP efficiency (Panel D).

Referring to FIG. 28, competitor oligonucleotide included in PCP reactions increases specificity of tagging. The competitor sequence is composed of the PCP receptor annealing sequence and so can anneal to the tagging RNA produced from the seed. The competitor will sequester tagging RNAs that diffuse from the seed, such that the molecules in proximity to the seed will be preferentially tagged in the PCP reaction. Nanobodies that are not bound to a Rap1 protein are not tagged by the PCP reaction due to the excess of competitor. The 3′ extremity of the competitor is blocked by a dideoxycytidine (ddC), preventing its elongation by reverse transcriptase in the PCP reaction.

Referring to FIG. 29, both the seed DNA and the nanobody receptor can be tagged in the PCP reaction and amplified by PCR. The amount of PCR product (analyzed by a native agarose gel) is proportional to the amount of specific tagging in the PCP. Each reaction has the same quantity of proteins, but differing numbers of ALFA epitope tags on each Rap1 protein. The amount of PCR product shows that nanobodies are tagged in proportion to the amount of ALFA epitope tags on the Rap1 protein.

### *****

### Equivalents

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A method of tagging one or more proximal molecules in a sample, the method comprising:
(i) admixing a seed nucleic acid, a receptor nucleic acid, an RNA polymerase, a reverse transcriptase, and two or more molecules to be tagged,
wherein the seed nucleic acid conjugates to a first site and comprises a promoter, a tag (or unique molecular identifier), and an annealing sequence,
wherein the receptor nucleic acid conjugates to a second site and comprises a nucleic acid sequence complementary to the annealing sequence
(ii) incubating the admixture for a period of time sufficient to allow
a. transcription of the seed nucleic acid by the RNA polymerase, thereby producing an RNA fragment,
b. annealing of the RNA to the receptor nucleic acid, and
c. reverse transcription of the RNA fragment by the reverse transcriptase, thereby producing a cDNA,
thereby tagging proximal molecules.

2. The method of claim 1, wherein the first site and the second site are on the same molecule, or wherein the first site and the second site are on different molecules.

3. The method of claim 1, wherein the promoter comprises a T7 promoter, T3 promoter, or an SP6 promoter.

4. The method of claim 1, wherein the annealing sequences comprises between 1 and 30 nucleotides.

5. The method of claim 4, wherein the annealing sequence comprises about 20 nucleotides.

6. The method of claim 1, wherein the annealing sequence comprises a nucleic acid sequence selected from the group consisting of 5′AAAAACCACAAAA 3′, 5′ AAAAGGAGAAAAAGGGAAAGAA 3′, 5′ AAAAGGAGAAAAAAAAGA 3′, 5′ AAAAGGAGAAAAA 3′, or 5′ TTTTGGTGTTTTT 3′.

7. The method of claim 1, wherein the seed nucleic acid, receptor nucleic acid, or both, comprises one or more modified ribonucleotide.

8. The method of claim 1, the receptor nucleic acid can be conjugated to a nanobody.

9. The method of claim 1, wherein the RNA polymerase comprises T7 or SP6.

10. The method of claim 1, wherein the reverse transcriptase is a recombinant M-MuLV reverse transcriptase.

11. The method of claim 1, wherein the annealing is dependent on the Tm of the complementary sequences.

12. The method of claim 1, wherein the method is carried out at between 20-37° C.

13. The method of claim 12, wherein the reaction is carried out at 30° C.

14. The method of claim 1, further comprising one or more molecular crowding agents.

15. The method of claim 14, wherein the molecular crowding agent comprises poly(ethylene glycol), glycerol, or both.

16. The method of claim 1, wherein the molecule is a nucleic acid, protein, or protein complex.

17. The method of claim 16, wherein the protein or protein complex comprises a histone or a nucleosome.

18. The method of claim 1, wherein the method comprises one or more additional steps of:
a. protein and/or DNA crosslinking prior to admixing; and/or
b. protein and/or DNA fragmentation prior to admixing; and/or
c. protein and/or DNA repair and A-tailing; and/or
d. seed nucleic acid and/or receptor nucleic acid ligation.

19. The method of claim 18, wherein the crosslinking agent comprises formaldehyde, disuccinimidyl glutarate, or both.

20. The method of claim 18, wherein the fragmentation comprises enzymatic fragmentation or mechanical fragmentation.

21. The method of claim 20, wherein enzymatic fragmentation comprises a nuclease.

22. The method of claim 18, wherein ligation agent comprises T4 DNA ligase.

23. The method of claim 1, wherein the method comprises one or more additional steps of:
a. a protein digestion step, and/or
b. an amplification step, and/or
c. a library preparation step, and/or
d. a sequencing step.

24. A kit comprising the reagents for any one of the methods of claim 1-23.

25. The kit of claim 24, wherein the kit comprises a seed nucleic acid, a receptor nucleic acid, an RNA polymerase, and a reverse transcriptase.

26. A method of whole chromosome analysis, wherein the method comprises the molecular tagging method of claim 1.

27. A method of mapping 3D genomic organization, wherein the method comprises the molecular tagging method of claim 1.

28. A method of protein detection or quantitation, wherein the method comprises the molecular tagging method of claim 1.
